(19) United States
(12) Patent Application Publication
(10) Pub. No.: US 2011/0245634 A1
(43) Pub. Date: Oct. 6, 2011

(54) Methods, Systems, and Devices for Analyzing Patient Data

(75) Inventors: Pinaki Ray, Fremont, CA (US); David Price, Pleasanton, CA (US)

(73) Assignee: LifeScan, Inc.

(21) Appl. No.: 13/078,711

(22) Filed: Apr. 1, 2011

Related U.S. Application Data
(60) Provisional application No. 61/320,701, filed on Apr. 3, 2010.

(51) Int. Cl. A61B 5/145

(52) U.S. Cl. 600/309

(57) ABSTRACT

Described herein is a method of analyzing an analyte distribution from discrete, quasi-continuous or continuous measurements to determine a glycemic state of a patient in order to understand how often, and for how long, a patient's post-prandial glucose is out of control without requiring laboratory blood test and especially post-prandial levels laboratory analysis. The systems, devices, and methods assist in predicting risk levels of developing diabetes-associated complications. Therefore applicants have recognized also a need for a tool which facilitates stratification of patients for risk of and/or onset of one or more complications having the same HbA1c level.

This application claims the benefits of priority under 35 USC §119 and/or §120 from prior filed U.S. Provisional Application Ser. No. 61/320,701 filed on Apr. 3, 2010, which applications are incorporated by reference in their entirety into this application.

### BACKGROUND

The incidence of diabetes is currently exploding worldwide. It is estimated that more than 44 million people in the United States alone are pre-diabetic, and unaware they have the condition. Diabetes results in a loss of control of blood sugar concentration. Complications from diabetes through loss of blood sugar control and in particular high blood sugars (hyperglycemia) can be debilitating and even life threatening. Health costs for treating such complications can be significant.

A proportion of patients suffering diabetes-related complications develop major health problems. Some people may be more prone to complications than others. This may be because of their lifestyle and/or for lack of blood glucose control. Currently it is not possible to predict who is most at risk of developing major health problems.

FIG. 25 shows a diagram of glucose ranges previously used in the diagnosis of diabetes and/or the prediction of a patient's risk of developing diabetes-related complications.

A common method used in screening for diabetes is the Fasting Plasma Glucose (FPG) test; a simple blood test taken after eight hours of fasting. As seen in FIG. 25A, a normal FPG range 2 is typically less than 100 mg/dl. A person with FPG values of >100 mg/dl on two different days is considered to be pre-diabetic having impaired fasting glycemia (IFG) 4 and potentially at risk of developing type 2 diabetes. A person with FPG levels of 126 mg/dl or above 6, measured on two different days indicates the presence of diabetes. This test may be confirmed by a second test, the oral glucose tolerance test (OGTT) whereby a blood test is taken two hours after ingestion of 75 grams of glucose. Patients with an OGTT level>140 mg/dl (see FIG. 25B) are considered to be pre-diabetic having an impaired glucose tolerance at 9, and those with an OGTT>200 mg/dl are considered to be diabetic level 7. Both FPG and post-prandial glucose impact glycated hemoglobin (HbA1C) which is itself an indicator of the risk of complications.

A groundbreaking study ‘The Diabetes Control and Complications Trial’ (DCCT) carried out over 9 years (1984-1993) and involving 1441 people with insulin-dependent diabetes throughout the USA and Canada, compared the effects of intensive and conventional insulin treatments on the development and progression of diabetic complications. Diabetics can be at risk of conditions associated with microvascular disease that can lead to cardiovascular disease, retinopathy (eye disease), neuropathy (nerve damage) and nephropathy (kidney disease). Other conditions associated with diabetes include circulatory problems, heart attacks and strokes.

Results from the DCCT study showed the lowest incidence of complications were found amongst those patients receiving intensive treatment (those having blood glucose levels averaging 8.6 mmol/l and glycated hemoglobin (HbA1c) levels of around 7%), compared to those in the conventional treatment group. HbA1c is a long term indicator of a patient's average blood sugar concentration, or long term glycemic condition typically over the previous two or three months. The DCCT and other similar studies have repeatedly demonstrated that the most effective way to prevent long-term diabetes-related complications is by strict control of blood glucose levels. Some adverse effects of intensive insulin treatment were however documented including increased risk of severe hypoglycemia and also weight gain. The risk of patients experiencing severe hypoglycemia forms a barrier to self-control of their condition.

In the past, some healthcare practitioners have taken the viewpoint that diabetic complications are inevitable and time, money and effort should not be spent on striving for good control of the condition. More recently, and in light of the DCCT study and others, efforts are increasingly concentrating on ways to achieve good diabetes control thereby reducing rates of complications and potentially delaying the onset of complications.

One way a diabetic patient's long-term ability to control and manage their condition may be monitored is by their glycated hemoglobin (HbA1c) value. However periods of high glucose concentrations may be balanced out by periods of low concentration, and patients seemingly under ‘good control’, as perceived by their HbA1c value, may have high blood sugars for small periods of time at certain times during the day, and these can easily go un-noticed. Patients with an HbA1c value indicative of good control may still have experienced damaging excursions and be at risk of associated complications. Another way to monitor a patient's glycemic condition is using self monitoring of blood glucose (SMBG) typically 1 to 3 times per day to indicate an immediate or current glycemic condition or glucose level in a subject's body at the time of measurement.

A limited number of patients use continuous systems that measure glucose concentration continuously or quasi-continuously e.g. every few minutes rather than intermittently a few times a day (previously on sale Cygnus measurement system, Free Style Navigator® Continuous Glucose Monitoring System from Abbot Diabetes Care, Alameda, Calif., GM SystemFDA approved DexCom™ STS™ Continuous Glucose Monitoring System available from DexCom Inc, San Diego, Calif., USA and Guardian RT™ Continuous Subcutaneous Glucose Monitoring System available from Medtronic Minimed, Northridge, Calif., USA).

Further, studies have indicated that post-prandial glucose is also a key risk factor in developing complications. It is known to measure 1,5 anhydroglucitol (1,5AG) as an intermediate measure of sensitivity to post-prandial glucose. A GLYCOMARK test available from GlycoMark Inc, Whisteon-Salem, N.C., USA, can be used to measure 1,5AG. The 1,5AG test gives an indication of the average post-prandial glucose levels over approximately the previous two weeks, with greatest influence from the most recent measurements e.g. previous two days. The 1,5AG measurement reflects an intermediate glycemic condition over an intermediate time period between HbA1c and intermittent blood glucose measurements. However, both HbA1c and 1,5AG tests require venipuncture and are carried out in a laboratory.

Hyperglycemia and elevated 1,5AG and/or HbA1c levels indicate increased risk of developing diabetes-related complications. To make daily management using SMBG more effective and practical, there is a need for a tool for use by patients and health care professionals (HCPs) that does not require a blood test. Furthermore, there is a need for a tool that provides a short/medium or intermediate term indicator of hyperglycemic excursions, and their impact, so intervention can occur quickly to reduce the hyperglycemia and hence risk of complications, and/or delay of onset. OGTT, which can be used to diagnose diabetes, is a hard glucose test to administer. Simply having a patient wear a continuous monitor patch for a few days and usage of a short-term indicator as described above can help detect abnormal glucose excursions (incl. fasting and post-prandial) and therefore lead to diagnosis of diabetes.

Dungan et al discussed 1,5 Anhydroglucitol and post-prandial hyperglycemia as measured in continuous glucose monitoring system in moderately controlled patients with diabetes in Diabetes Care, Volume 29, Number 6, June 2006, p. 1214. More recently, Mazze et al have discussed characterizing glucose exposure for individuals with normal glucose tolerance using continuous glucose monitoring and ambulatory glucose profiles (Diabetes Technology and Therapeutics, Volume 10, Number 3, 2008, p. 149). Mazze has discussed the future of self-monitored blood glucose: mean blood glucose versus glycosated hemoglobin in Diabetes Technology and Therapeutics, Volume 10, Supplement 1, p. S-93. As continuous glucose measurement (CGM) techniques improve applicants anticipate that more people will adopt such technologies. Applicants have recognized therefore a need for data analysis tools and methodologies that can span both technological approaches to analyte measurement (intermittent and continuous), enabling a patient's transition from one to the other. Moreover applicants have recognized a need for tools for use with both SMBG and CGM that can supplement glycemic indicators, including HbA1c and 1,5AG and other indicators known to those skilled in the art. Further, applicants have recognized a need for tools for use with both technologies which provide intermediate timescale glycemic assessment. This may reduce the need for clinic based tests such as venipuncture as required by an HbA1c test. Without such tools, data management in continuous monitoring could present a barrier to uptake.

Furthermore, it is important to understand how often, and for how long, a patient's post-prandial glucose is out of control. Applicants have recognized a need for monitoring tools that respond rapidly to occurrence of high blood sugar levels. Therefore also applicants have recognized a need for monitoring tools that do not require a laboratory blood test and especially post-prandial levels laboratory analysis.

There is currently no commercially available analytical tool that determines whether a patient is more prone to complications than a patient with the same glycemic characterization value such as HbA1c or 1,5AG or other characteristic value, using only blood glucose data. Applicants have recognized therefore a need for such a tool.

Furthermore, applicants have recognized a need for tools which assist in predicting risk levels of developing diabetes-associated complications. Therefore applicants have recognized also a need for a tool which facilitates stratification of patients for risk of and/or onset of one or more complications having the same HbA1c level.

Based on their experience, Health Care Professionals (HCPs), in a practice, hospital or region may be able to estimate the likelihood of diabetes complications from an FPG or OGTT tests. Nevertheless, these tests can be inconvenient for the patient and HCP to repeat for the purpose of monitoring risk of complications. Firstly, patients have to plan to visit their HCP practitioner. Secondly, they have to fast for eight hours, and thirdly, a blood test is required which can be painful or uncomfortable or cause distress. Furthermore, interaction and communication between the HCP and the patient is required following the tests which can take up HCP's time. Such estimations can be valuable but these can be subjective and vary across several HCP practices or hospitals or regions. Applicants have recognized therefore a need for an analytical tool that can estimate risk of complications across several HCPs, HCP practices, one or more hospitals, regions or populations without the need for an HCP supervised blood test.

### SUMMARY OF THE DISCLOSURE

In one aspect of the invention there is provided a method of analyzing an analyte distribution from discrete, quasi-continuous or continuous measurements. The method can be achieved by: defining a predetermined time period T; collecting n analyte measurements Gi each associated with a time ti within predetermined time period T with each analyte measurement by a transformation of analyte disposed in body fluid into an enzymatic by-product disposed in body fluid into an enzymatic by-product; repeating step (b) for N predetermined time periods T; aggregating the analyte measurements Gi to determine the number of occurrences of each value of Gi across N predetermined time periods T; fitting a curve y=f(Gi) to the number of occurrences versus the value of G to determine an estimated probability y=f(G) of the value G occurring within any given predetermined time period Ti.

In a further aspect of the invention there is provided a method of analyzing an analyte distribution from discrete, quasi-continuous or continuous measurements. The method can be achieved by: defining a predetermined time period T; providing a measuring device to collect n analyte measurements Gi in a body fluid each associated with a time ti within predetermined time period T with each analyte measurement by a transformation of analyte disposed in body fluid into an enzymatic by-product disposed in body fluid into an enzymatic by-product; repeating the step of collecting at step (b) for N predetermined time periods T; providing a microprocessor adapted to aggregate the analyte measurements Gi to determine the number of occurrences of each value of Gi across N predetermined time periods T; operating a microprocessor to fit a curve y=f(Gi) to the number of occurrences versus the value of G to determine an estimated probability y=f(G) of the value G occurring within any given predetermined time period Ti; determining for a user an estimated probability of occurrence of at least one value of G from the fitted curve.

In a further aspect of the invention, there is provided a device for analyzing an analyte distribution from discrete, quasi-continuous or continuous measurements. The device has a collector to collect analyte measurements Gi and a microprocessor to receive the analyte measurements. The microprocessor is programmed to carry out the following instructions: defining a predetermined time period T; collecting n analyte measurements Gᵢ each associated with a time tᵢ within predetermined time period T with each analyte measurement by a transformation of analyte disposed in body fluid into an enzymatic by-product disposed in body fluid into an enzymatic by-product; repeating step (b) for N predetermined time periods T; aggregating the analyte measurements Gᵢ to determine the number of occurrences of each value of Gᵢ across N predetermined time periods T; fitting a curve y=f(Gᵢ) to the number of occurrences versus the value of G to determine an estimated probability y=f(G) of the value G occurring within any given predetermined time period Tᵢ; and determining for a user an estimated probability or occurrence of at least one value of G from the fitted curve.

In yet another aspect, a method of estimating a value of a patient characteristic from an analyte distribution of discrete, quasi-continuous or continuous measurements from a patient is provided. The method can be achieved by: defining a predetermined time period T; collecting n analyte measurements Gᵢ each associated with a time t, within predetermined time period T with each analyte measurement by a transformation of analyte disposed in body fluid into an enzymatic by-product disposed in body fluid into an enzymatic by-product; repeating step (b) for N predetermined time periods T; aggregating the analyte measurements Gᵢ to determine the number of occurrences of each value of Gᵢ across N predetermined time periods T; fitting a curve y=f(Gᵢ) to the number of occurrences versus the value of G to determine an estimated probability y=f(G) of the value of G occurring within any given time period T for a first patient for a first series of predetermined time periods N₁; determining a lower limit Lᵢ, of analyte measurement G; calculating a first area A₁¹ under a first fitted probability curve above the lower limit L₁, representing the probability of measurement G occurring above lower limit L₁; measuring a value of said patient's characteristic C₁^{m}; determining a first relation between said first area A₁¹ and said measured characteristic C₁^{m}; repeating steps (a) to (e) for a second series of predetermined time periods N₂; calculating a second area A₁² under a second fitted probability curve above the lower limit L₁; and using the relation between first area A₁¹ and first measured characteristic C₁^{m} and the second area A₁² to provide an estimate value of said patient's characteristic C₂^{est} during said second series of time periods N₂.

In another method of estimating a value of a patient characteristic from an analyte distribution of discrete, quasi-continuous or continuous measurements from a patient. The method can be achieved by: defining a predetermined time period T; providing a measuring device to collect n analyte measurements Gᵢ in a body fluid each associated with a time tᵢ within predetermined time period T with each analyte measurement by a transformation of analyte disposed in body fluid into an enzymatic by-product disposed in body fluid into an enzymatic by-product; repeating the step of collecting at step (b) for N predetermined time periods T; providing a microprocessor adapted to aggregate the analyte measurements Gᵢ to determine the number of occurrences of each value of Gᵢ across N predetermined time periods T; operating the microprocessor to fit a curve y=f(Gᵢ) to the number of occurrences versus the value of G to determine an estimated probability y=f(G) of the value of G occurring within any given time period T for a first patient for a first series of predetermined time periods N₁; determining a lower limit L₁, of analyte measurement G; calculating a first area A₁¹ under a first fitted probability curve above the lower limit L₁, representing the probability of measurement G occurring above lower limit L₁; measuring a value of said patient's characteristic C₁^{m}; determining a first relation between said first area A₁¹ and said measured characteristic C₁^{m}; repeating steps (a) to (e) for a second series of predetermined time periods N₂; calculating a second area A₁² under a second fitted probability curve above the lower limit L₁; operating the microprocessor to use the relation between first area A₁¹ and first measured characteristic C₁^{m} and the second area A₁² to determine an estimate value of said patient's characteristic C₂^{est} during said second series of time periods N₂ and to provide same to a user.

In a further method of estimating a Standard Excursion Area associated with a patient characteristic from an analyte distribution from discrete, quasi-continuous or continuous data from a cohort of patients. The method can be achieved by: defining a predetermined time period T; collecting n analyte measurements Gᵢ each associated with a time tᵢ within predetermined time period T with each analyte measurement by a transformation of analyte disposed in body fluid into an enzymatic by-product disposed in body fluid into an enzymatic by-product; repeating step (b) for N predetermined time periods T; aggregating the analyte measurements Gᵢ to determine the number of occurrences of each value of Gᵢ across N predetermined time periods T; fitting a curve y=f(Gᵢ) to the number of occurrences versus the value of G to determine an estimated probability y=f(G) of G occurring within any given time period T for a first patient for a first series of predetermined time periods N₁; determining a lower limit L₁, of an analyte measurement G; for each patient determining a first area A₁¹ under a fitted probability curve above the lower limit L₁; for each patient measuring a value of said first characteristic C₁^{m}; selecting first areas A¹₁ and grouping these into at least one group according to a value or a range of values of the measured characteristic C^{m}₁; for at least one group, determining a First Standard Excursion Area from the first areas A¹₁ within that group, the First Standard Excursion Area being associated with the values of the characteristic for that group for that lower limit.

In still another method of estimating a Standard Excursion Area associated with a patient characteristic from an analyte distribution from discrete, quasi-continuous or continuous data from a cohort of patients. The method can be achieved by: defining a predetermined time period T; providing a measuring device to collect n analyte measurements Gᵢ in a body fluid each associated with a time tᵢ within predetermined time period T with each analyte measurement by a transformation of analyte disposed in body fluid into an enzymatic by-product disposed in body fluid into an enzymatic by-product; repeating the step of collecting at step (b) for N predetermined time periods T; providing a microprocessor adapted to aggregate the analyte measurements Gᵢ to determine the number of occurrences of each value of Gᵢ across N predetermined time periods T; operating the microprocessor to fit a curve y=f(Gᵢ) to the number of occurrences versus the value of G to determine an estimated probability y=f(G) of G occurring within any given time period T for a first patient for a first series of predetermined time periods N₁; determining a lower limit L₁, of an analyte measurement G; for each patient determining a first area A₁¹ under a fitted probability curve above the lower limit L₁; for each patient measuring a value of said first characteristic C₁^{m}; selecting first areas A¹₁ and grouping these into at least one group according to a value or a range of values of the measured characteristic C^{m}₁; for at least one group, determining for a user a First Standard Excursion Area from the first areas A¹₁ within that group, the First Standard Excursion Area being associated with the values of the characteristic for that group for that lower limit.

In another aspect, a method of estimating a patient characteristic is provided. The method can be achieved by: determining a patient specific excursion area Aₚ; retrieving at least one Standard Excursion Area Aₛ for the characteristic; comparing the patient specific excursion area Aₚ with the Standard Excursion Area Aₛ, and providing an estimate of a patient characteristic to a user from the comparison.

In still yet a further aspect, a method of analyzing a condition, characteristic, complication or risk thereof for a cohort of patients. The method can be achieved by: determining at least one lower limit L₁ and at least one Standard Excursion Area As for a specific condition, characteristic or complication or risk thereof; determining probability density curves by analyte measurement for each patient in a cohort; operating a microprocessor to determine the excursion area Aₚ¹ under the probability density curve above the at least one lower limit L₁ for each patient in a cohort; operating the microprocessor to compare the patient specific excursion areas Aₚ¹ with the Standard Excursion Area to provide to a user an estimate of the patient's condition, characteristic or complication Cₚ^{est} or risk thereof Risk (Cₚ^{est}) for patients in the cohort.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements). A detailed understanding of the features and advantages of the present invention will be obtained by reference to the following detailed description that sets forth illustrative embodiments, in which the principles of the invention are utilized, and the accompanying drawings of which:

FIG. 1A illustrates a diabetes management system that includes an analyte measurement and data management unit and data communication devices.

FIG. 1B illustrates, in simplified schematic, an exemplary circuit board of a diabetes data management unit.

FIG. 2 shows a flow diagram of a process of collecting data and using same in an analytical tool according to an example embodiment in a first aspect of the invention;

FIGS. 3A to 3D show theoretical example self-monitoring blood glucose measurements over 3 days and an associated probability density plot and fitted curve according to an example embodiment of a first aspect of the invention;

FIGS. 4A to 4D shows theoretical example continuous blood glucose monitoring measurements over 3 days and an associated probability density curve and fitted curve according to an example embodiment of a first aspect of the invention;

FIG. 5 shows process steps associated with an example embodiment of a first aspect of the invention;

FIG. 6 shows further process steps in an analytical tool according to an example embodiment of a second aspect of the invention;

FIG. 7 shows further process steps in an analytical tool according to a further example embodiment of a second aspect of the invention;

FIG. 8 shows further process steps of an analytical tool according to a further example embodiment of a second aspect of the invention;

FIG. 9 shows optional process steps of further example embodiments according to a second aspect of the invention;

FIG. 10 shows optional process steps which can be used in any of the embodiments of any aspect of the present invention;

FIG. 11 shows a table detailing previously determined correlations between 1,5AG values, blood glucose concentrations and a health assessment statement indicative of a patient's level of glycemic control (see US patent application US20080187943);

FIG. 12 shows a table giving correlations between HbA1_{c} values, 1,5AG values and excursion area values Aⁿ_{EXC}, according to an example embodiment in a third aspect of the invention; Similar ranges could be identified by those skilled in art for other characteristics e.g., fructosamine, cholesterol;

FIG. 13 shows a general method of determining a First Standard Excursion Area associated with a given health risk according to a fourth aspect of the invention;

FIG. 14 shows a specific method of determining a Standard Excursion Area for relating analyte measurements, here blood glucose concentration measurements, to a pre-defined characteristic, here glycemic control assessment (e; g; 1,5AG range);

FIG. 15 shows a method of determining the glycemic control assessment for an individual patient using the relationship between Standard Excursion Area and pre-defined glycemic control assessment for example as derived in FIG. 14; Optional process steps for use in any embodiment of the invention are also shown;

FIG. 16 shows optional process steps for use in a general method of determining second or further standard Excursion Areas and associating same with different health conditions, characteristics, complications or risks thereof according to a further example embodiment of a fourth aspect of this invention;

FIG. 17 shows a method of determining a risk associated with a given patient for a condition, characteristic or complication Y in an example embodiment according to a fifth aspect of the invention; Optional steps which can be used in any embodiment of this invention are also shown;

FIG. 18 shows a method of conducting a stratification of patients using process steps from FIGS. 13 to 16 and process steps of FIG. 17 for a cohort of patients in a first example embodiment according to a sixth aspect of the invention;

FIG. 19 shows a method of stratifying patients for a cohort of patients in further example embodiments according to a sixth aspect of the invention; Optional process steps which can be used in any embodiment of this invention are also shown;

FIG. 20 shows a graph of fitted curve to frequency of example SMBG data against glucose value, showing a post-meal high glucose threshold (140 mg/dl according to American Diabetes Association);

FIG. 21 shows a graph of fitted curve to frequency of example SMBG data versus glucose value, showing two excursion areas with glycemic thresholds defined for two different types of condition (here diabetes complications);

FIGS. 22A and 22B show example plots of probability density curves comparing patients over several weeks with higher and lower levels of risk of developing disease related complications for (a) post-prandial hyperglycemia and (b) high fasting plasma glucose;

FIG. 23 shows optional process steps to determine a merit ratio according to an example embodiment according to a seventh aspect of the invention; and

FIG. 24 shows a table of examples of figures of merit M and associated quality as a figure of merit using the previously determined correlations shown in FIG. 23 in a further example embodiment according to a seventh aspect of the invention.

FIG. 25A and FIG. 25B show respective glucose ranges typically used in Fasting Plasma Glucose (FPG) test and Oral Glucose Tolerance Test (OGTT) for diagnosing diabetes and pre-diabetes;

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment. For simplicity, the word ‘characteristic’ is used herein to indicate a condition, characteristic, complication or risk of a condition, characteristic or complication and it should be taken to mean that were used. Here, “excursion” means a movement of a level of glucose from an acceptable value to an unacceptable value, and back again to a normal value. For simplicity, the invention is discussed in relation to an example characteristic, such as 1,5AG. Other suitable characteristics include HbA1c, fructosamine and others in the common general knowledge of those skilled in the art. Furthermore, also for simplicity, the invention is discussed in relation to a particular analyte such as glucose. Other analytes may be monitored, such as ketones, cholesterol, and fructosamine, as would be understood by those skilled in the art.

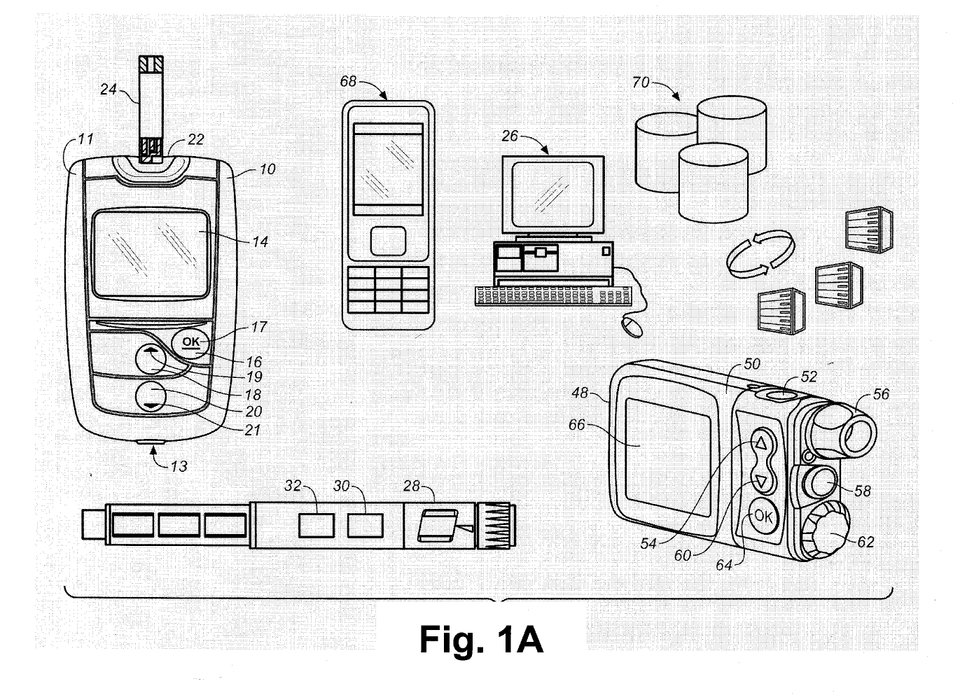

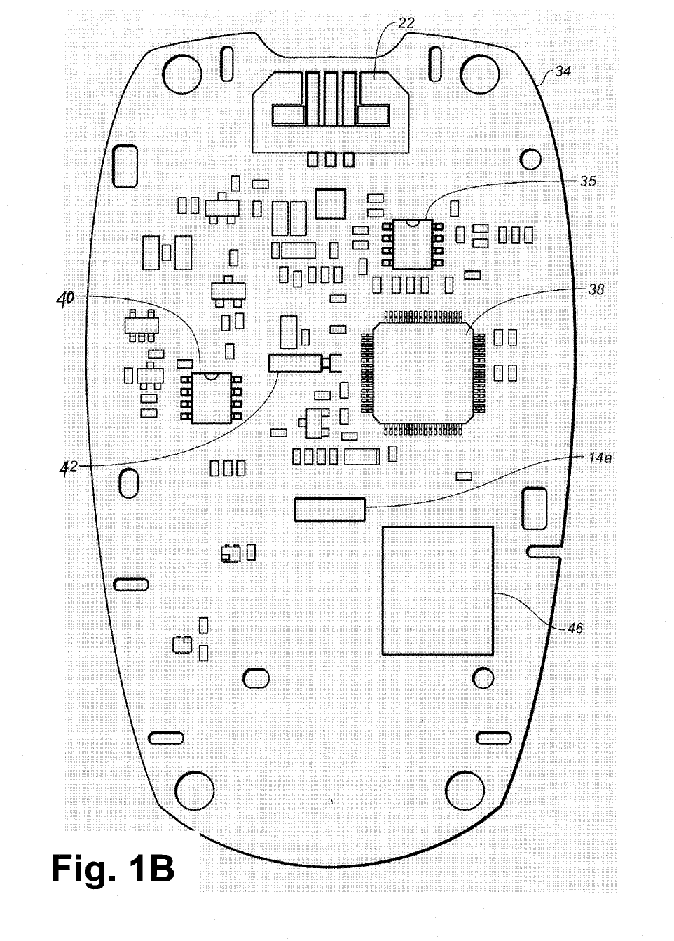

FIG. 1A illustrates a diabetes management system that includes an analyte measurement and management unit 10, therapeutic dosing devices (28 or 48), and data/communication devices (68, 26, or 70). Analyte measurement and management unit 10 can be configured to wirelessly communicate with a handheld glucose-insulin data management unit or DMU such as, for example, an insulin pen 28, an insulin pump 48, a mobile phone 68, or through a combination of the exemplary handheld glucose-insulin data management unit devices in communication with a personal computer 26 or network server 70, as described herein. As used herein, the nomenclature “DMU” represents either individual unit 10, 28, 48, 68, separately or all of the handheld glucose-insulin data management units (28, 48, 68) usable together in a disease management system. Further, the analyte measurement and management unit or DMU 10 is intended to include a glucose meter, a meter, an analyte measurement device, an insulin delivery device or a combination of an analyte testing and drug delivery device. In an embodiment, analyte measurement and management unit 10 may be connected to personal computer 26 with a cable. In an alternative, the DMU may be connected to the computer 26 or server 70 via a suitable wireless technology such as, for example, GSM, CDMA, BlueTooth, WiFi and the like.

Glucose meter or DMU 10 can include a housing 11, user interface buttons (16, 18, and 20), a display 14, a strip port connector 22, and a data port 13, as illustrated in FIG. 1A. User interface buttons (16, 18, and 20) can be configured to allow the entry of data, navigation of menus, and execution of commands. Data can include values representative of analyte concentration, and/or information, which are related to the everyday lifestyle of an individual. Information, which is related to the everyday lifestyle, can include food intake, medication use, occurrence of health check-ups, and general health condition and exercise levels of an individual. Specifically, user interface buttons (16, 18, and 20) include a first user interface button 16, a second user interface button 18, and a third user interface button 20. User interface buttons (16, 18, and 20) include a first marking 17, a second marking 19, and a third marking 21, respectively, which allow a user to navigate through the user interface.

The electronic components of meter 10 can be disposed on a circuit board 34 that is within housing 11. FIG. 1B illustrates (in simplified schematic form) the electronic components disposed on a top surface (not shown) of circuit board 34, respectively. On the top surface, the electronic components include a strip port connector 22, an operational amplifier circuit 35, a microcontroller 38, a display connector 14a, a non-volatile memory 40, a clock 42, and a first wireless module 46. Microcontroller 38 can be electrically connected to strip port connector 22, operational amplifier circuit 35, first wireless module 46, display 14, non-volatile memory 40, clock 42, and user interface buttons (16, 18, and 20).

Operational amplifier circuit 35 can include two or more operational amplifiers configured to provide a portion of the potentiostat function and the current measurement function. The potentiostat function can refer to the application of a test voltage between at least two electrodes of a test strip. The current function can refer to the measurement of a test current resulting from the applied test voltage. The current measurement may be performed with a current-to-voltage converter. Microcontroller 38 can be in the form of a mixed signal microprocessor (MSP) such as, for example, the Texas Instrument MSP 430. The MSP 430 can be configured to also perform a portion of the potentiostat function and the current measurement function. In addition, the MSP 430 can also include volatile and non-volatile memory. In another embodiment, many of the electronic components can be integrated with the microcontroller in the form of an application specific integrated circuit (ASIC).

Strip port connector 22 can be configured to form an electrical connection to the test strip. Display connector 14a can be configured to attach to display 14. Display 14 can be in the form of a liquid crystal display for reporting measured glucose levels, and for facilitating entry of lifestyle related information. Display 14 can optionally include a backlight. A data port can be provided to accept a suitable connector attached to a connecting lead, thereby allowing glucose meter 10 to be linked to an external device such as a personal computer. The data port can be any port that allows for transmission of data such as, for example, a serial, USB, or a parallel port. Clock 42 can be configured to keep current time related to the geographic region in which the user is located and also to measure time. The DMU can be configured to be electrically connected to a power supply such as, for example, a battery.

In one exemplary embodiment, test strip 24 can be in the form of an electrochemical glucose test strip. Test strip 24 can include one or more working electrodes and a counter electrode. Test strip 24 can also include a plurality of electrical contact pads, where each electrode can be in electrical communication with at least one electrical contact pad. Strip port connector 22 can be configured to electrically interface to the electrical contact pads and form electrical communication with the electrodes. Test strip 24 can include a reagent layer that is disposed over at least one electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). An exemplary mediator suitable for use in the reagent layer includes ferricyanide, which in this case is in the oxidized form. The reagent layer can be configured to physically transform glucose into an enzymatic by-product and in the process generate an amount of reduced mediator (e.g., ferrocyanide) that is proportional to the glucose concentration. The working electrode can then measure a concentration of the reduced mediator in the form of a current. In turn, glucose meter 10 can convert the current magnitude into a glucose concentration. Details of the preferred test strip are provided in U.S. Pat. Nos. 6,179,979; 6,193,873; 6,284,125; 6,413,410; 6,475,372; 6,716,577; 6,749,887; 6,863,801; 6,890,421; 7,045,046; 7,291,256; 7,498,132, all of which are incorporated by reference in their entireties herein.

Referring back to FIG. 1A, insulin pen 28 can include a housing, preferably elongated and of sufficient size to be handled by a human hand comfortably. The device 28 can be provided with an electronic module 30 to record dosage amounts delivered by the user. The device 28 may include a second wireless module 32 disposed in the housing that, automatically without prompting from a user, transmits a signal to first wireless module 46 of the DMU 10. The wireless signal can include, in an exemplary embodiment, data to (a) type of therapeutic agent delivered; (b) amount of therapeutic agent delivered to the user; or (c) time and date of therapeutic agent delivery.

In one embodiment, a therapeutic delivery device can be in the form of a “user-activated” therapeutic delivery device, which requires a manual interaction between the device and a user (for example, by a user pushing a button on the device) to initiate a single therapeutic agent delivery event and that in the absence of such manual interaction delivers no therapeutic agent to the user. A non-limiting example of such a user-activated therapeutic agent delivery device is described in co-pending U.S. Non-Provisional application Ser. Nos. 12/407,173 (tentatively identified by Attorney Docket No. LFS-5180USNP); 12/417875 (tentatively identified by Attorney Docket No. LFS-5183USNP); and 12/540217 (tentatively identified by Attorney Docket No. DDI-5176USNP), which is hereby incorporated in whole by reference. Another non-limiting example of such a user-activated therapeutic agent delivery device is an insulin pen 28. Insulin pens can be loaded with a vial or cartridge of insulin, and can be attached to a disposable needle. Portions of the insulin pen can be reusable, or the insulin pen can be completely disposable. Insulin pens are commercially available from companies such as Novo Nordisk, Aventis, and Eli Lilly, and can be used with a variety of insulin, such as Novolog, Humalog, Levemir, and Lantus.

Referring to FIG. 1A, a therapeutic dosing device can also be a pump 48 that includes a housing 50, a backlight button 52, an up button 54, a cartridge cap 56, a bolus button 58, a down button 60, a battery cap 62, an OK button 64, and a display 66. Pump 48 can be configured to dispense medication such as, for example, insulin for regulating glucose levels.

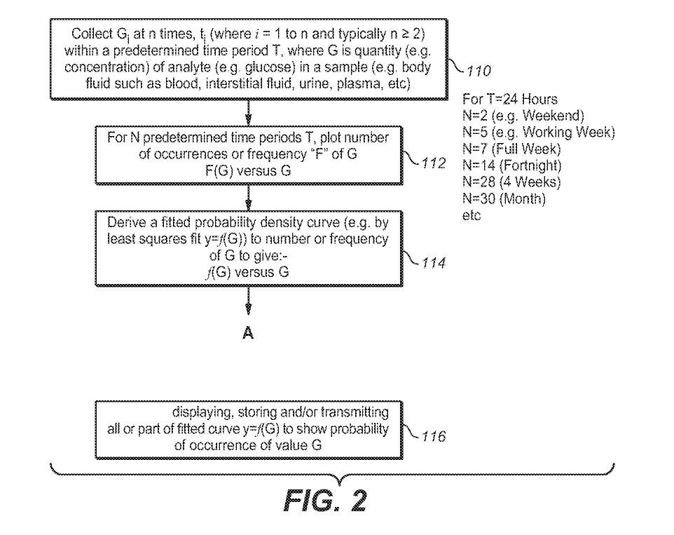

As will be discussed in more detail in relation to FIGS. 2 to 9, analytical tools for use in embodiments described herein are described. Relatively high frequency self-monitoring of blood glucose is required for input to the analytical tool(s), for example, 3 or more tests per day, and alternatively, some or all taken when fasting and/or post-prandially.

Firstly, self-monitoring blood glucose (SMBG) data or continuous data is collected over a pre-defined collection period. Next, the occurrences (or frequency) of blood glucose readings against pre-defined glucose ranges are transformed into the range of readings obtained during the data collection period. Next, a probability-density curve is fitted to each distribution for example, using software incorporating the method of the present invention. The area under the curve, described above, for example a defined post-meal target glucose concentration threshold, is calculated by integration. In one example embodiment of the invention, this fitted curve is displayed in a display (on a meter, pc, PDA, phone etc) finally, this provides an indicator of the patient's excursion above the threshold e.g., Post Meal Target, if post-prandial measurements are collected.

A relatively high level of SMBG monitoring is required to achieve a high level of control. It is recommended that at least 3 post-prandial blood glucose measurements are taken for each patient each day of each week. This may be a higher frequency of testing than is perceived as normal for many diabetics, however it is a relatively simple means to gain tighter control of the disease, and minimize the risk of future complications. Measurements collected over a set period of time, for example one week, are loaded into a particular processor (e.g. in a meter, pc, PDA, phone etc) and analyzed. Frequency y=f(G) of occurrence of measurements is transformed against predefined glucose ranges (G), and a probability-density curve fitted to the data to allow the user or HCP to view a reflection of the true physiologic state of the patient.

In one example embodiment, the analytical tool disclosed involves determining the glucose excursion area for each patient each week. Exposure of tissue cells to high blood glucose frequently and/or for prolonged periods can be debilitating, and lead to life-threatening health complications. The excursion area provides an indication of how often and for how long the patient experienced hyperglycemic excursions during each week, and can be used as an index to determine the risk of complications due to such post-prandial excursions. The excursion area and the risk of complications are thought to be more or less proportional i.e., the higher the number of readings in the hyperglycemic range; the higher the area calculated predicting a greater risk of complications.

It is intended that this analytical tool and associated software is for use by healthcare practitioners, and may be loaded onto and run by desktop computer or workstation, or a hand-held computer. It would be apparent to those skilled in the art that alternative devices such as, including a particular machine or purpose-built portable meter, additional software for existing conventional blood glucose meters, personal digital assistants, phones and the like may also be used.

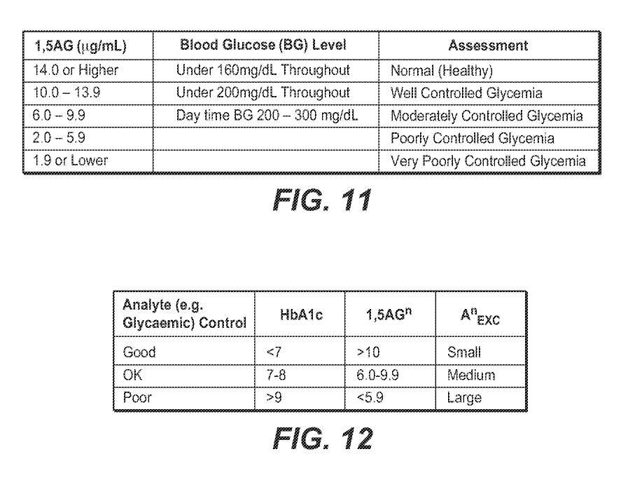

Furthermore, in one example embodiment an estimate of a patient's level of glycemic control by prediction of fructosamine, HbA1c or 1,5AG, values or other glycemic indicators from SMBG data is determined. For convenience, this will now be discussed in relation to 1,5AG and SMBG; however, this approach can equally be used with Hb1Ac, fructosamine and other indicator values of glycemic condition, or indeed analytes other than glucose and for other conditions. In an example embodiment, if the analytical tool is being used for a patient for the first time, then it will be useful for correlation purposes to also determine a 1,5AG value for the patient. The excursion area determined for the patient can then be associated with this 1,5AG value, and a standard excursion area to 1,5AG ratio (or relationship) for the patient is thus obtained. Excursion areas calculated from SMBG values for subsequent weeks can then be used in conjunction with the standard ratio/relationship to predict weekly 1,5AG values (without the need for venipuncture), allowing an assessment of the patients diabetes control using an already established scale (see FIG. 11). Alternatively, several 1,5AG tests may be carried out for further comparative purposes.

In more detail now, FIG. 2 shows a flow diagram of a method of collecting and analyzing data from discrete, quasi-continuous or continuous measurements. At step 110, the method comprises collecting measurements Gi of an analyte or indicator n times, at time ti. where i=1 to n. Typically, n will be greater than or equal to 2 and alternatively, greater than or equal to 3 in any given predetermined time period T. Gi is a quantity of an analyte or indicator that can be measured e.g., presence, concentration, density, viscosity and so on of an analyte or indicator such as glucose, ketones, cholesterol, proteins, phenylamine or enzymes and so on (for example, glucose concentration) in a liquid sample, for example, body fluid such as blood, interstitial fluid, urine, plasma, saliva etc. G represents a quantity of analyte at time t. Thus Gi is a discrete quantity representing the value at points in time of a continuously varying quantity G. Hereinafter, analyte is used for simplicity, nevertheless where analyte is referred to it is to be understood to mean any suitable analyte or indicator. Likewise, hereinafter, blood is used for simplicity nevertheless where blood is referred to it is to be understood to mean any suitable body fluid such as blood, interstitial fluid, plasma, urine, saliva and the like.

In FIG. 2 at step 112, for a total of N predetermined time periods T, the number of occurrences or frequency of Gi are transformed into a histogram F(G). Thus F(G) is a graph of the number of occurrences of G or frequency of G versus G. This aggregation and plotting of analyte measurements Gi can be carried out for discrete measurements, such as discrete self-monitoring of blood glucose concentration (e.g. 3 times per day), quasi-continuous measurements (e.g. every 1-30 minutes) or truly continuous measurements (analogue). Next, at step 114, a curve y=f(G) is fitted to the histogram F(G). This fitting may be carried out by any suitable method such as least squares fitting, linear regression, or other methods known to those skilled in the art.

Thus, steps 110, 112, 114 enable an estimated probability density curve y=f(G), for the probability of occurrence of any given value of G, to be determined.

In discrete measurements, such as self monitoring of blood glucose (SMBG), the number of measurements Gᵢ in any given time period T is likely to be low and irregularly spaced. Indeed although measurements G taken under an SMBG regime may follow a general pattern each day, these are nevertheless somewhat randomly spaced.

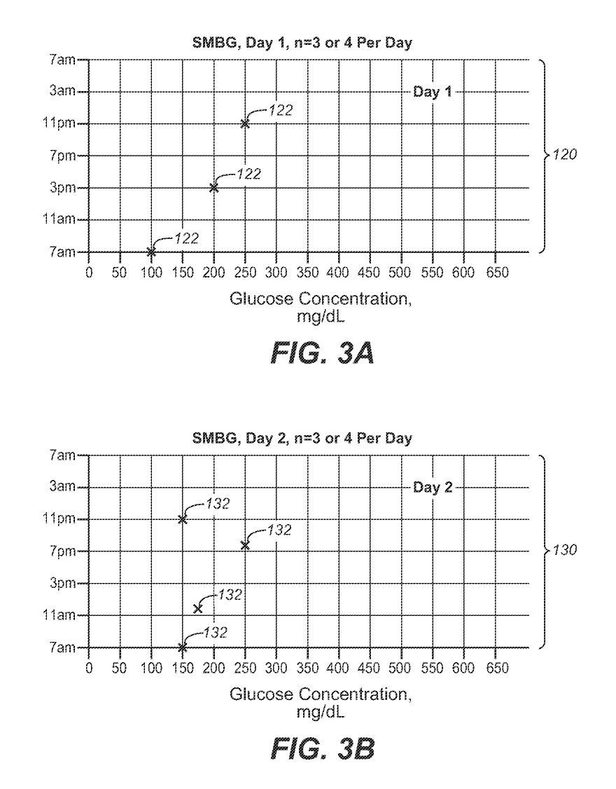

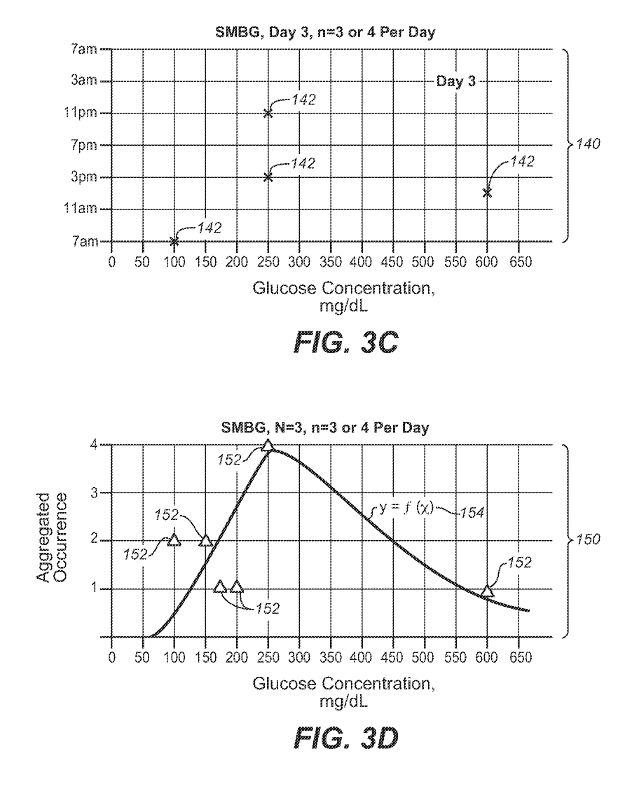

Over several days or weeks as further measurements are added, an SMBG histogram plot will more accurately reflect the average transit of blood glucose concentration of the patient over a day. By contrast, in quasi-continuous or continuous measurements, such as continuous measurement of blood glucose concentration, measurements Gᵢ are likely to be frequently and regularly collected and equi-spaced. The present invention enables each source of these types of data to be similarly used to provide an estimate of the probability density of an analyte measurement G within a given time period. This will be discussed in more detail in relation to FIGS. 3 & 4.

Indeed for both discrete and non-discrete continuous measurements, when N is low, the probability curve for occurrence of value G will be less accurate than when N is large. N can equal any suitable value such as one selected from the group of 2, 5, 7, 14, 28, 30, 60, 90, 120 representing 2 days, 5 days etc. While it is convenient to use days as units of N, any other convenient time period can be used such as an hour, half days and so on.

Alternatively, at step 116 of FIG. 2, part or all of the fitted probability density curve y=f(G) can be displayed to a user such as a patient or healthcare professional to indicate probability of occurrence of a range of values G based on past measurements.

In more detail now, FIGS. 3A to 3D show a schematic representation of discrete intermittent analyte measurements e.g. SMBG. Here, example self-monitoring blood glucose data for N=3, i.e., 3 days worth of measurement, T=24 hours or 1 day, and n=3 or 4 per period T (i.e., per day) are shown. During day 1, as shown in graph 120, 3 measurements 122 of blood glucose concentration are taken at 7 am, 3 pm and 11 pm. On day 2, as shown in graph 130, four measurements 132 are taken at 7 am, 11.30 am, 7.30 am and 11 pm. In day 3, as shown in graph 140, four measurements 142 taken at 7 am, noon, 3 pm and 11 pm. These three days worth of results are aggregated to provide a histogram showing frequency or number of measurements G in graph 150 to give aggregated occurrence values 152. A curve y=f(x) where x=G i.e., y=f(G) 154 has been fitted to points 152. This curve represents the probability density of a given analyte measurement G occurring within predetermined time period T (in this case 1 day). As more days' data is added, this probability curve 154 becomes a more accurate representation of the probability.

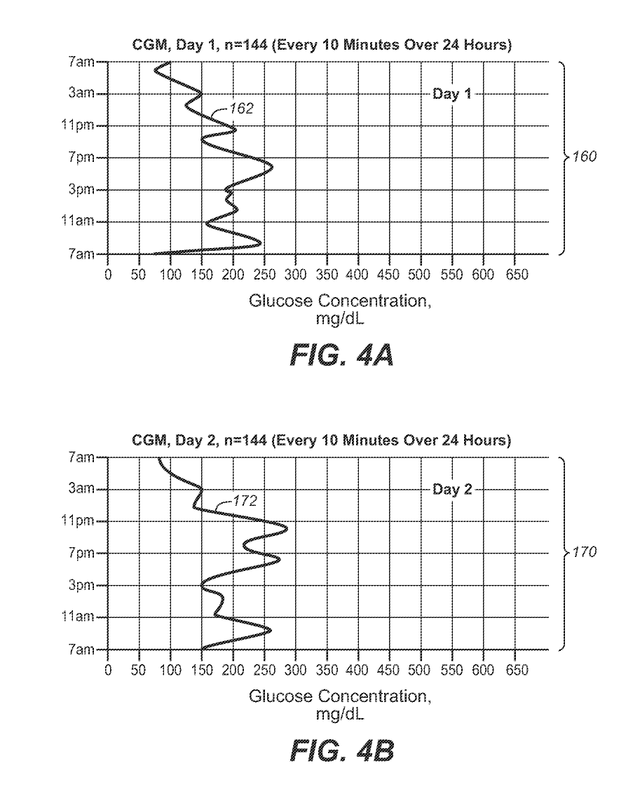

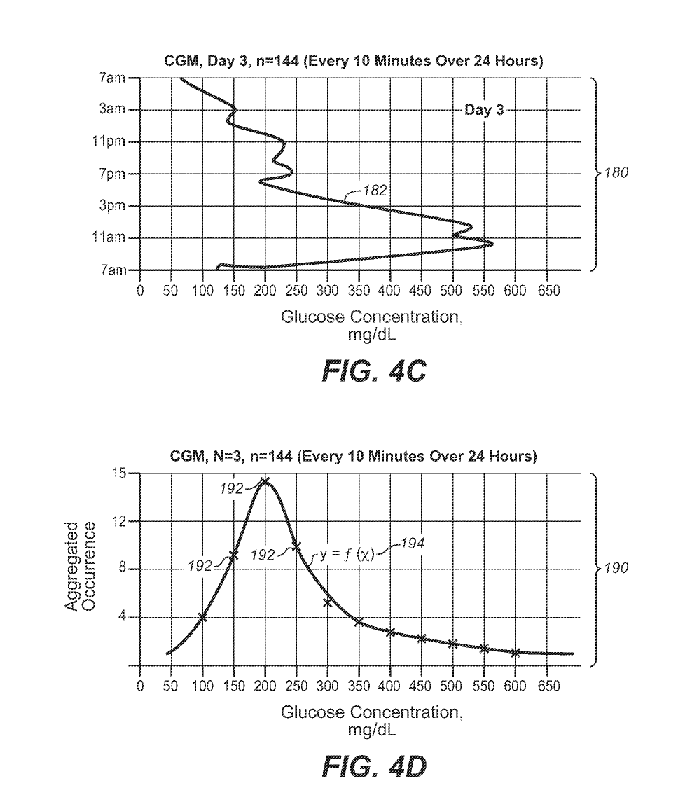

FIG. 4A to 4D show a schematic representation of 3 days worth of example quasi-continuous glucose measurements taken over 3 days (N=3) every 10 minutes, for T=24 hours or per day, giving a total number of measurements in every predetermined time period T of n=144.

In day 1, as shown in graph 160 a series of measurements 162 of value G are taken. Similarly, during days 2 and 3 as shown in graphs 170 and 180, a series of values 172 and 182 are measured. These results are aggregated by summing or averaging as shown in graph 190 to provide a frequency plot of data points 192. A curve y=f(x) 194 is fitted to data points 192 to provide the probability density curve of value G. Thus one can estimate the probability of occurrence of a value of G (along the x axis) by using the relation y=f(G) to determine the probability of that value of G.

As described in relation to FIG. 2 and the graphs in FIGS. 3 and 4, it has been shown that both discrete and continuous (or quasi-continuous) data can be analyzed and displayed in a similar manner. There are some differences however. Whereas in continuous analyte measurements each measurement is given the same weight and is spaced evenly throughout the predetermined time period T this is not the case with discrete analyte measurements. Thus, there is an assumption in FIG. 3 that each of the 3 or 4 measurements during each predetermined time period are evenly spaced and therefore should be weighted evenly. This assumption is not always accurate for SMBG data for example if data points 122, 132 or 142 are taken close together. Typically, however, discrete measurements are spaced throughout the predetermined time period somewhat randomly. The inventors have appreciated that although data may not be regularly spaced, these are typically sufficiently well spaced as to provide a good enough approximation. In an alternative embodiment, discrete data points can be flagged, for example; post-prandially, and/or during fasting, so that only these measurements are used in the analysis. Indeed, post-prandial measurements are likely to be approximately evenly spaced throughout the waking part of an individual's day since these follow meal times and therefore selecting such results for analysis further ensures that this analysis is based on a reasonable spread of results throughout the day. This further provides benefit in that such measurements post-prandially are thought to provide insight into a patient's movement within a condition such as diabetes and are therefore particularly useful to look at. Thus the present invention is used to transform real data, representing real physical characteristics from disparate sources, into a probability of time spent or predicted probability of occurrence of a real physical characteristic. Given appropriate safeguards, this estimate of a real physical characteristic can be used to make changes to behavior, medication doses, food intake, exercise, etc.

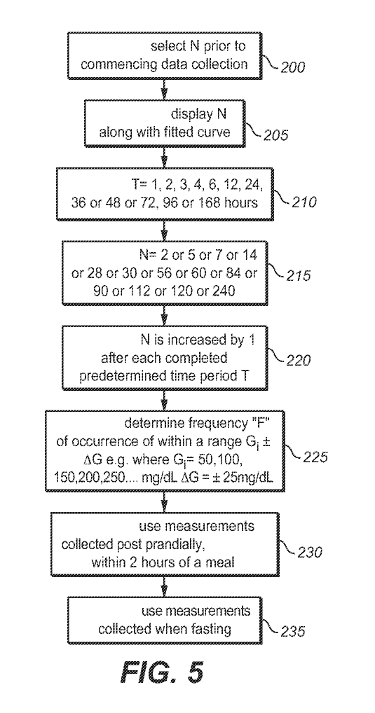

Referring now to FIG. 5, in further embodiment of a first aspect of the invention, step 200 shows that N, the number of predetermined time periods T, can be selected prior to commencing data collection. At step 205, alternatively, N is displayed along with part or all of the fitted probability density curve from step 116.

Step 210 indicates that while T can be any suitable value, alternatively, T can be selected from the group of 1, 2, 3, 4, 6, 12, 24, 36, 48, 72, 96, 168 hours. Step 215 indicates that while N can be any suitable value, Alternatively, N can be selected from the group of N=2, 5, 7, 14, 28, 30, 56, 60, 84, 90, 112, 120, 240. Optional step 220 indicates that N can be increased by 1 after each completed predetermined time period. This would enable a running total of analyte measurements to be used in the determination of the probability density curve, this total increasing by 1 after completion of each time period T (e.g. a day). Thus, the user can wait for, for example, a week between updates of each probability density curve, or, the probability density curve can be updated each day using N days worth of data on one day, N+1 days worth of data on the next day, N+2 days worth of data on the next day and so on. Step 225 indicates that the number or frequency of occurrence of G can be within a range e.g. G±ΔG. This can be particularly useful in discrete measurement analysis enabling the formation of a traditional histogram. Typically, where measurements G are blood glucose concentration the range of ±ΔG can be ±2, 5, 10, 15, 20, 25, 50 mg/dL when G is measured in mg/dL. Step 230 and 235 indicate that, alternatively, postprandial and/or fasting measurements only may be used to provide the analysis.

In continuous analyte measurement monitoring, such as continuous glucose monitoring, the measurements are typically evenly spaced throughout the day. Because the total number of measurements over one day is much higher than in discrete measurements such as SMBG, the number of days required to obtain suitable quantities of data to form an accurate probability density plot is less. Over time, over sufficient number of days, (i.e., over sufficient predetermined time periods T) the probability density curve from discrete measurements will approach that from continuous measurements as more and more data is collected. Nevertheless, the preferred embodiments allow a common analytical, transformation tool to be used whatever the nature of the source data. This tool provides the ability to educate a patient to understand data presented in a common way and can be very useful for a patient who is moving from discrete measurement to continuous measurement. Similarly it can enable a healthcare provider to use a common analytical tool for all patients no matter what measurement technology and regime they use. Thus, this embodiment provides a tool for use in methods and devices that allows common comparison of data from different patients, or for patients moving across measurement regimes.

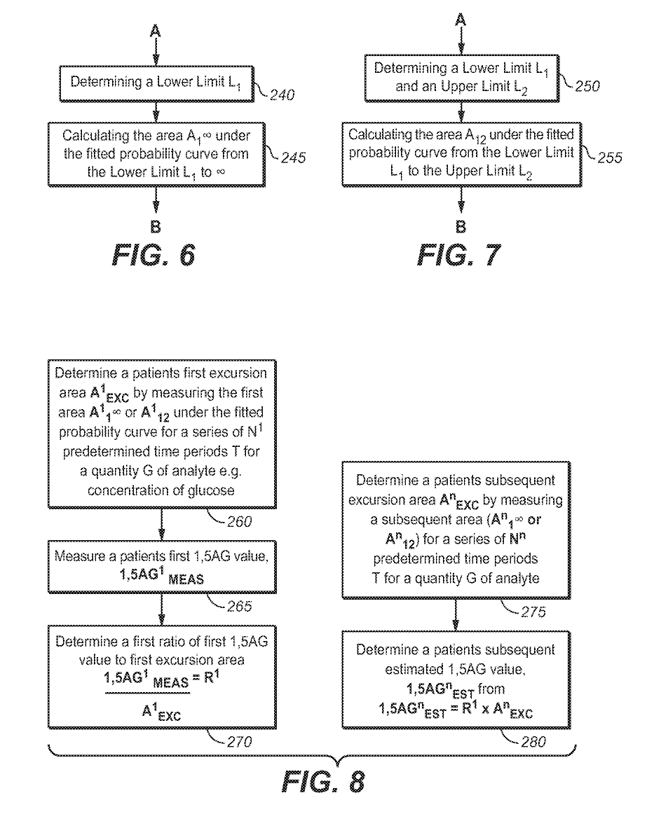

In a second embodiment as shown in FIG. 6 an area under the fitted probability density curve can be calculated. Thus, at step 240 a lower threshold limit L₁ is determined. At step 245 an area A_{1∞} under the fitted probability density curve can be calculated from the lower limit L₁ to ∞. In an alternative embodiment, the area under the fitted probability curve can be calculated between a lower limit L₁ and an upper limit L₂ as shown at steps 250 and 255 of FIG. 7.

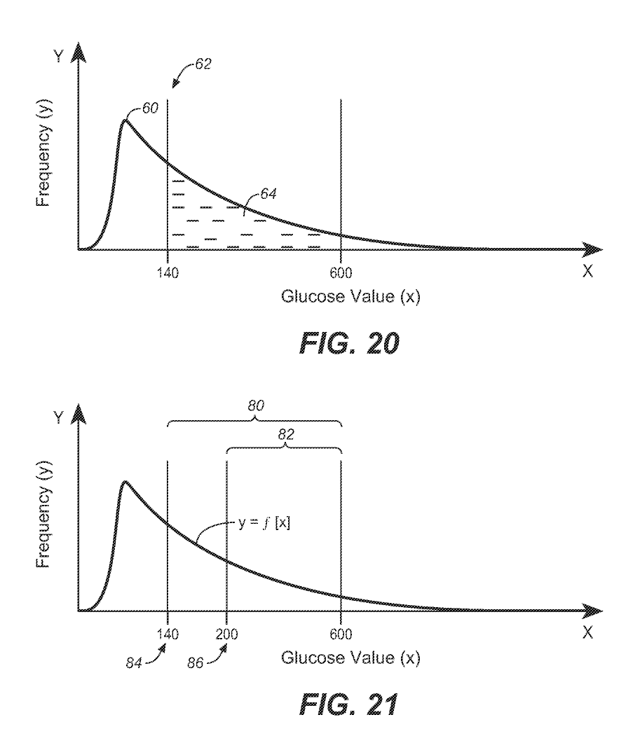

Referring briefly now to FIG. 20 there is shown a graph of a typical SMBG data distribution, showing a probability density curve 60 with a lower threshold limit 62 for a post-meal glucose high threshold and an excursion area 64. FIG. 20 shows, by way of example, a frequency versus glucose range plot that could be obtained using blood glucose data from a patient over the course of one week. Individual data values are not shown, only the probability-density curve 60 obtained by non-linear regression through an example data set plotted as a histogram. Each probability density curve 60 will be slightly different for the same patient at different times or for different patients, but may follow the typical shape or pattern outlined in FIG. 20. Excursion area 64 can be determined between lower threshold limit 62 and co or between a lower and an upper threshold limit (say between 140 mg/dL and 600 mg/dL). Although a period of one week is described other periods of testing may also be utilized.

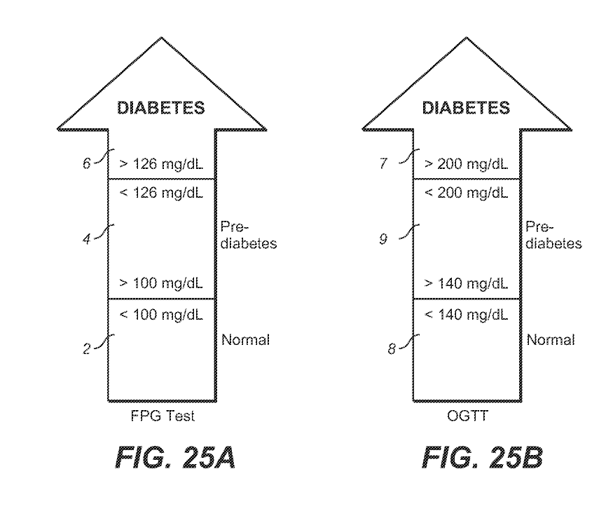

As described in FIG. 25B, blood glucose values below 140 mg/dL are considered normal, and many of the readings may fall below this value. The American Diabetes Association (ADA) defines the threshold limit 62 for post meal glucose as blood glucose concentrations having a value less than 140 mg/dL. Patients having blood glucose concentrations>140 mg/dL post meal are potentially at pre- risk of developing diabetes.

The excursion area 64 for each patient, each week, Alternatively, is obtained by integrating the area under the curve between a threshold limit such as threshold limit 62, for example 140 mg/dl and a maximum value, for example 600 mg/dL as at steps 245 and 255 of FIGS. 6 and 7 to give:

Excursion area=∫₁₄₀⁶⁰⁰f(x)dx

Referring now to FIG. 8, a method of estimating a patient characteristic C₁^{m} from distribution of a patient's measurements is described in more detail. Firstly, at step 260, the patient's first excursion area A¹_{EXC} is determined by measuring the first area A¹_{1∫}, or A¹₁₂ as described above in relation to FIGS. 6 and 7 for a series of N¹ predetermined time periods T for a quantity G of analyte e.g. concentration of glucose in a body fluid. A patient's first characteristic value C₁^{m} is measured at step 265 at approximately the same time as step 260. In this example embodiment, the characteristic value is a 1,5AG value. Other characteristic values can be used in this and other embodiments such as HbA1c, fructosamine and other characteristics known to those skilled in the art.

At step 270 a first relation R¹ (such as a ratio for example) of first characteristic value, such as 1,5AG to first excursion area, is determined. At step 275, the patient's subsequent excursion area Aⁿ_{EXC} is determined by measuring a subsequent excursion area A¹_{1∫} or Aⁿ₁₂ for a series of Nⁿ predetermined time periods for a quantity G of analyte. Next, at step 280, a patient's subsequent characteristic value, such as 1,5AG, is estimated from the relation between the first characteristic value and the first excursion area as determined at step 270. Typically, the relation will be a ratio as shown at steps 270 and 280. Where the relation is not a ratio, this may be determined by linear regression or other method.

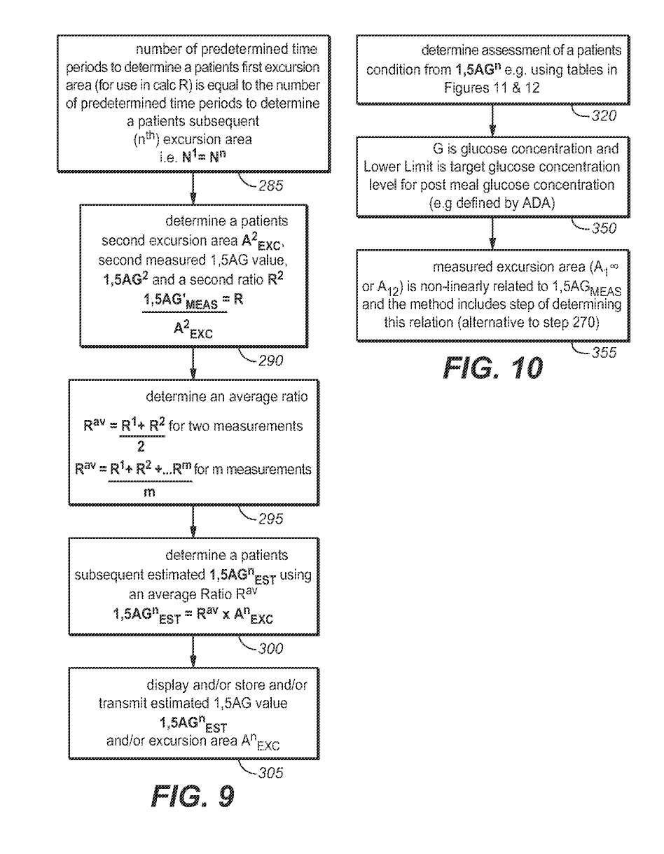

FIG. 9 shows several optional steps for the estimation of a patient characteristic from an analyte concentration distribution of a patient. Alternatively, the number N¹ of predetermined time periods T to determine a patient's first excursion area and its relation to a patients characteristic is equal to the number Nⁿ of predetermined time periods T to determine a patient's subsequent n^{th} excursion area. This need not necessarily be the case. Typically T¹ and Tⁿ are equal, although this need not be the case.

At step 290, a patient's second excursion area and second measured characteristic value such as 1,5AG value are used to determine a second relation between one another, for example a second ratio R².

As can be seen at step 295, an average of the relation, such as the ratio, can be determined. For example an average ratio R^{av} can be determined from a first ratio R¹ and second ratio of R² or from several ratios such as R¹ to R^{m} averaged over m measurements of such ratios. Alternatively, as at step 300, this average relation (e.g. ratio) can be used to determine the estimated characteristic value from the latest excursion area. Use of an average relation, such as a ratio, is likely to prove more accurate over time as more data is collected.

At step 305, the estimated characteristic value such as 1,5AG value can be displayed and/or stored and/or transmitted as required by the user or the healthcare professional.

FIG. 10 shows optional process steps which can be used in any of the embodiments of any aspect of the present invention.

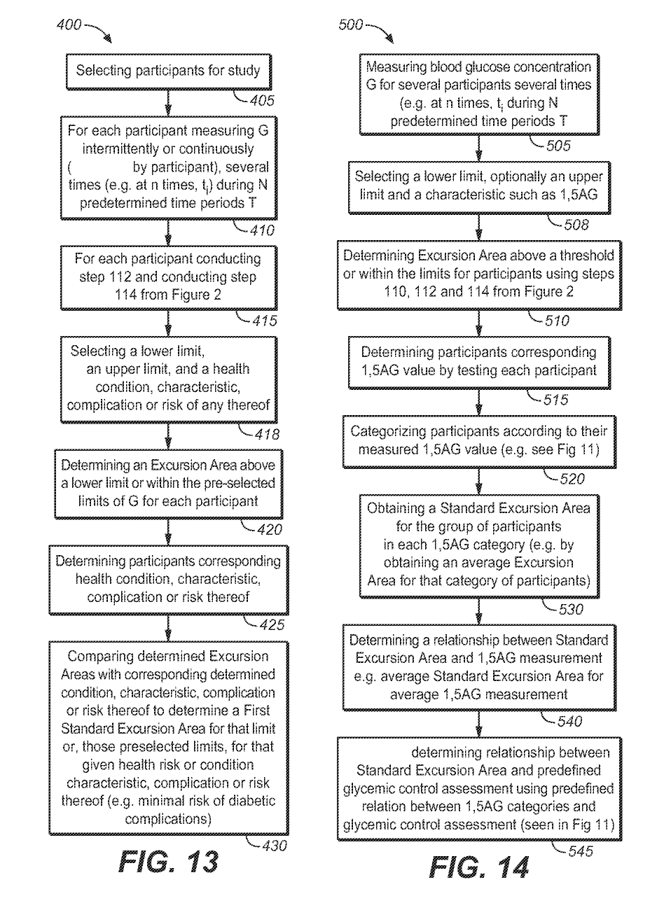

FIG. 10 shows at step 320, alternatively, determining assessment of a patient's condition (e.g. glycemic control) using FIGS. 11 and 12. Further, at step 350 Alternatively, G is glucose concentration and the lower limit is the target glucose concentration level for post meal (post-prandial) glucose concentration.

At step 355, the measured excursion area is non-linearly related to the characteristic and any of the methods of the invention includes the step of determining this relation.

FIG. 11 shows a table giving previously determined correlations between 1,5AG values, blood glucose concentrations and a health assessment statement indicative of a patient's level of glycemic control. Thus, the table indicates the relationship between average blood glucose level, a characteristic value 1,5AG and health risk assessment, for example, a rating of 1,5AG value of 14.0 or higher would indicate a normal (healthy) glycemic control assessment.

Having determined a relationship between excursion area and 1,5AG value for each patient as described in relation to FIGS. 6 and 7, this can then be used to predict future HbA1c or 1,5AG values from SMBG data, as described in relation to FIG. 8. By using an index of (intermediate) glycemic control currently available with regard to HbA1c (or 1,5AG as in FIG. 11), a relationship between (intermediate) glycemic control and SMBG data is therefore provided by certain aspects of the present invention.

Use of SMBG as an indicator of intermediate glycemic control ensures that patients and/or HCPs have an easily available measure of the patient's intermediate glycemic control, in comparison to conventional 1,5AG or HbA1c measurements. Furthermore, patients and/or HCPs have a measure of glycemic control that can be updated as required (daily, weekly, and monthly). Furthermore, blood glucose concentrations measured many times each day will better monitor short transients in glucose excursions, often un-noticed by other testing methods. Detailed knowledge of a patient's glycemic control allows physicians, and/or the patient themselves, to better understand factors affecting their level of control. This is believed by applicants to allow for patients to better understand and even reduce the risk of developing further disease-related complications, thereby alleviating some of the pressure on the healthcare system and insurance providers.

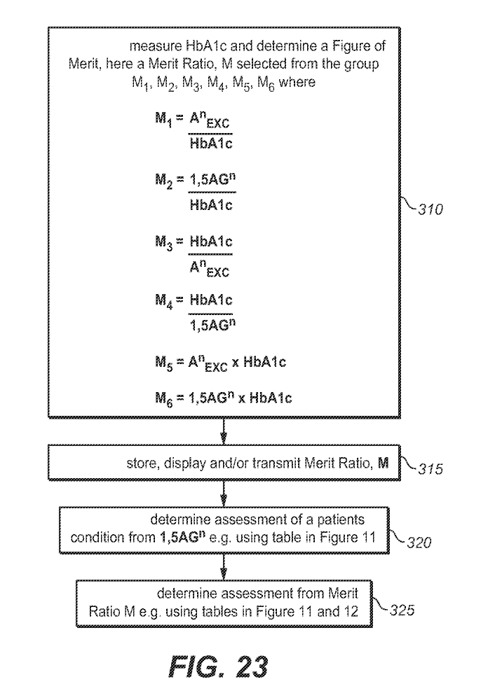

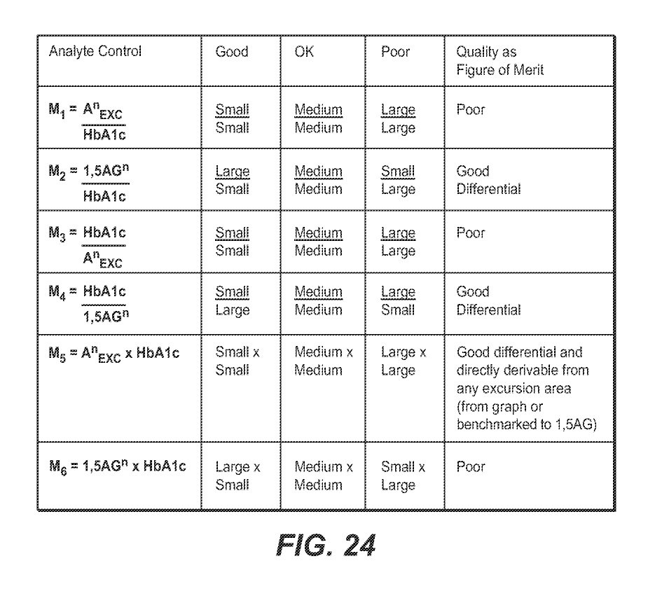

Referring briefly to FIG. 23, this figure shows optional determination of a figure of merit M selected from the group M₁, M₂, . . . M₆ where M is as shown at step 310. Figures of merit can be used to stratify patients. Selection of an appropriate figure of merit can enhance the spread of patients across the strata making stratification easier. At step 315 the figure of merit M is stored, displayed and/or transmitted as required. Step 320 indicates optional assessment and determination of a patient's condition from a characteristic value e.g.; HbA1c or 1,5AG using a table such as that shown in FIG. 11. Alternatively, as shown at step 325 a patient's assessment can be carried out using a merit ratio M (where M=M₁, M₂, M₃, M₄, M₅ or M₆ as shown in FIG. 23). Alternatively, in one example preferred embodiment M=M₂ or M₄ or M₅ or more preferably, M=M₅ (see FIGS. 23 and 24).

FIG. 12 shows, for a variety of characteristics, the ranges over which these characteristics are indicative of good, acceptable or poor analyte (e.g. glycemic) control. The characteristics shown are HbA1c, excursion area Aⁿ_{EXC} and 1,5AGⁿ. Thus, good glycemic control is indicated if the HbA1c value is less than 7%, the excursion area is small, or the 1,5AG value is greater than 10, whereas poor glycemic control is indicated if the HbA1c value is greater than 9, the excursion area is large or the 1,5AG value is less than 5.9.

FIG. 24 shows each of the suggested figures of merit M₁ to M₆ and their relative quality as a figure of merit based upon the differential for good, ok or poor analyte control as shown in FIG. 12. Thus, figure of merit M₁ (ratio of excursion area to HbA1c measurement) is a relatively poor figure of merit since at good levels of analyte e.g. glycemic control, the HbA1c value and the excursion value are both small and therefore their ratio tends towards 1 at all levels of analyte control. This is in contrast to figures of merit M₂, M₄ and M₅ which show a good differential between the ratios or factors at all levels of analyte control. In particular, figure of merit M₅, which is excursion area multiplied by HbA1c value, shows good differential between good analyte control, acceptable analyte control and poor analyte control. In addition, figure of merit M₅ is proportional to any excursion area on any fitted probability density curve as previously discussed, for the same HbA1c level.

FIGS. 13 & 14 describe the steps involved in generating a Golden Standard excursion area, by means of a clinical study involving a cross-section of the diabetic population. The process typically requires the involvement of a broad cross-section of the diabetic population for a clinical trial. Each participant is required to frequently or continuously measure their blood glucose concentration. Corresponding HbA1c, 1,5AG or other characteristic values are also required, and these can be measured directly. The excursion area above a predetermined threshold value (e.g. 140 mg/dL) is then determined for each participant, and involves the calculation of a ‘Standard’ excursion area, through standardization of the data. Such a Standard Excursion Area can then be used as a means for quickly determining whether a patient is at risk of developing complications related to their diabetes.

Through clinical trials involving a number of diabetics, including the capture of personal information such as age, sex, lifestyle etc., as well as health-related information, it is possible to determine ‘Standard Excursion Areas’ either for each category of glycemic control provided in FIG. 11, and/or potentially define one ‘golden standard’ excursion area (similar to the 7% value for HbA1c). Such a golden standard could then be used by physicians to quickly differentiate between those patients at risk of developing disease-related complications from those who are less at risk. A ‘golden standard’ excursion area value would give the standardized, or generic indicator level of exposure time of tissue cells to periods of high glucose concentration, known from extensive studies to cause damage. A standard value, as described herein, could be used by physicians and the like, to gain a quick indication of a patient's short-term glycemic control and hence their ability to self-manage their diabetes. Provision of such an analytical tool may also facilitate physicians with a means for making a decision quickly, which would otherwise be delayed for several days or weeks in the wait for results from laboratory tests.

In more detail now FIG. 13 shows a method of conducting a clinical study 400 according to a further aspect of the invention for relating a First Standard Excursion Area with Specific health risk or condition, characteristic or complication. The method comprises a first step 405 of selecting participants for the study. At step 410, for each participant, an analyte measurement G is measured, alternatively, by the participants, several times during N predetermined time periods T. Alternatively, continuous measurements of G can be taken. At step 415, these measurements are used to determine a frequency or number of occurrences of measurements G as at step 112. Next, a fitted probability density curve is derived from the number of occurrences of G as at step 114.

Next, at step 420, an excursion area (area under the fitted probability density curve), above a predetermined threshold limit of G is determined for each participant. Next, at step 425, a participant's known corresponding condition or health risk such as diabetes, pre-diabetes, risk of diabetes complications, glycemic control, risk of cardiovascular disease, risk of renal failure etc is determined. Next, at step 430, the determined excursion areas are compared with the known condition or health risk from step 425 to determine a First Standard Exclusion Area for that threshold limit associated with that condition or health risk.

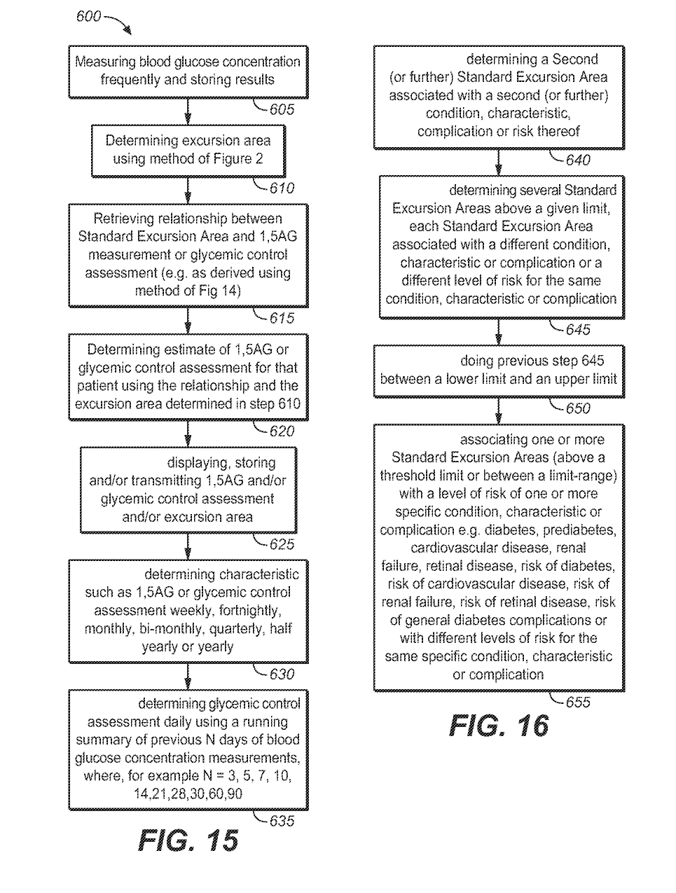

In more detail now, FIG. 14 shows a method of conducting a clinical study for relating a Standard Excursion Area to glycemic control using blood glucose measurements and characteristic such as HbA1c or 1,5AG etc. At step 505, blood glucose concentration is measured frequently for each participant. Alternatively, continuous measurements of G can be taken. At step 508, a lower limit, alternatively, an upper limit and a characteristic such as 1,5AG are selected. At step 510, an excursion area above a predetermined threshold value for one or more predetermined threshold limits, or between one or more predetermined limits is calculated for each participant. At step 515, corresponding characteristics e.g. 1,5AG, values are determined by testing. At step 520 patients are categorized according to their measured 1,5AG values and the 1,5AG categories are associated with predefined glycemic control assessment (see FIG. 11). At step 530, a Standard Excursion Area for each category of patients is obtained e.g. by obtaining an average Excursion Area for that category of patients having that characteristic value C₁^{m}. At step 540, a relation is determined between Standard Excursion Areas and predefined glycemic control assessment (such as that seen in FIG. 11) for one or more predetermined threshold limits, or between one or more predetermined limits. Thus, Standard (or Golden) Excursion areas can be determined.

FIG. 15 shows the method 600 for determining the glycemic control assessment for a specific patient. At step 605 blood glucose concentration is measured frequently and the results are stored. At step 610 an excursion area above a predetermined limit (or between predetermined limits) is determined. At step 615 the relationship, such as a ratio, between Standard Excursion Area and predefined glycemic control assessment, previously derived e.g. in a study as in FIG. 14, is retrieved. This retrieved relation may be from an existing memory e.g. from portable monitoring device, pda, dongle, personal computer or other memory device, from a remote central database or from a healthcare professional. At step 620, the retrieved relationship is used to determine the glycemic control assessment for the patient. Alternatively, as at step 625, the glycemic control assessment and/or excursion area is displayed and/or stored and/or transmitted using phrases such as OK, GOOD, POOR, or IN RANGE, HIGH, LOW or IN RANGE, OUT OF RANGE or HIGH RISK, LOW RISK, ACCEPTABLE RISK and so on. Such a transmission could be via a healthcare professional. At step 630, alternatively, glycemic control assessment takes place weekly, fortnightly, monthly etc. Alternatively, as at step 630, glycemic control assessment is determined daily using a running summary of the previous N days of blood glucose measurements. For example, N could be 3, 5, 7, 10, 14, 21, 28, 30, 60, and 90.

FIG. 16 shows additional process steps involved in a further embodiment of the invention. In general now this embodiment of the invention involves the establishment of standard glycemic threshold limits for specific known types of complications. For example 140 mg/dL may be established as the standard threshold value for cardiovascular complications, and 200 mg/dL for high risk of renal and/or retinol diseases, again determined via clinical trials or studies. These limits are selected as the upper target value of a desirable glycemic range before expected onset of that type of complication. Furthermore, standardized excursion areas outside of the target areas for each different type of complication will also be determined by means of clinical trials. While specific target threshold limits are mentioned, it will be apparent that alternative target threshold limits can be used.

The steps of FIG. 16 involve clinical trials whereby each participant has a known diabetes-related health complication. In an alternative embodiment, standard glycemic threshold values can be determined for each type of complication. From these, Standard Excursion Areas can be determined for one or more types of complication. Next a patient's excursion area is calculated. In this example embodiment, the threshold values are specific to certain types of diabetes-related health complications. The excursion area(s) for one or more complication can then be calculated for each patient, and compared against the Standard Excursion Areas developed through the clinical study i.e., the level of exposure of tissue cells to high glucose concentration known to cause such complications. Risk-ratios of developing specific complications can then be calculated for each patient, which can be monitored by their HCP. Risk-ratios can be used by HCPs to identify those patients at greatest risk of developing each complication.

Such a method outlines the use of clinical studies to determine high-risk threshold values for each type of diabetes-related complication. Standard Excursion Areas above each threshold value are then characterized for each type of complication. The probability-density curves obtained for patients, and the excursion areas above the threshold values for specific complications can then be calculated for each patient. Several excursion areas may be calculated for a patient, depending on the threshold value used, and compared against the standard high-risk excursion areas (determined by clinical trial for each type of complication). A risk ratio can then be calculated for each patient for each type of complication.

Risk ratios provide an indication of which patients are at high risk of developing certain complications, helping healthcare practitioners to identify them quickly and focus resources.

In more detail now, FIG. 16 shows several optional steps for any method of the invention. Step 640 shows the step of alternatively, determining a second (or further) excursion area associated with (a second) or (further) health risk. Step 645 shows alternatively, determining one or more Standard Excursion Areas for a lower threshold limit, each associated with a level of health risk.

Step 650 shows, alternatively, doing step 645 between a lower threshold limit and an upper limit. Step 655 shows doing one or both of step 645 or step 650 and associating one or more Standard Excursion Areas above a threshold limit or between a limit range with a level of health risk of one or more specific conditions e.g.; diagnosing diabetes, general diabetes complications, retinal disease, cardiovascular disease, renal failure etc.

FIGS. 13 to 16 outlines possible methods of determining a Standard Excursion Area, utilizing knowledge of both HbA1c or 1,5AG etc and frequent SMBG or continuous blood glucose data for each participant taking part in a clinical trial. The excursion area above a threshold value of, for example 140 mg/dL (as defined by the ADA as the ‘post-meal high-glucose’) is calculated for groups of patients fitting within a number of categories e.g. the five described in FIG. 11, as determined via their corresponding e.g. 1,5AG measurements. For each of the five categories, a number of excursion areas will then be obtained from the participants, from which a typical Standardized Excursion Area for that category, plus/minus a standard deviation can be obtained. The condition, characteristic or complication or risk thereof for that group of patients can then be associated with the Standard Excursion Area for that category.

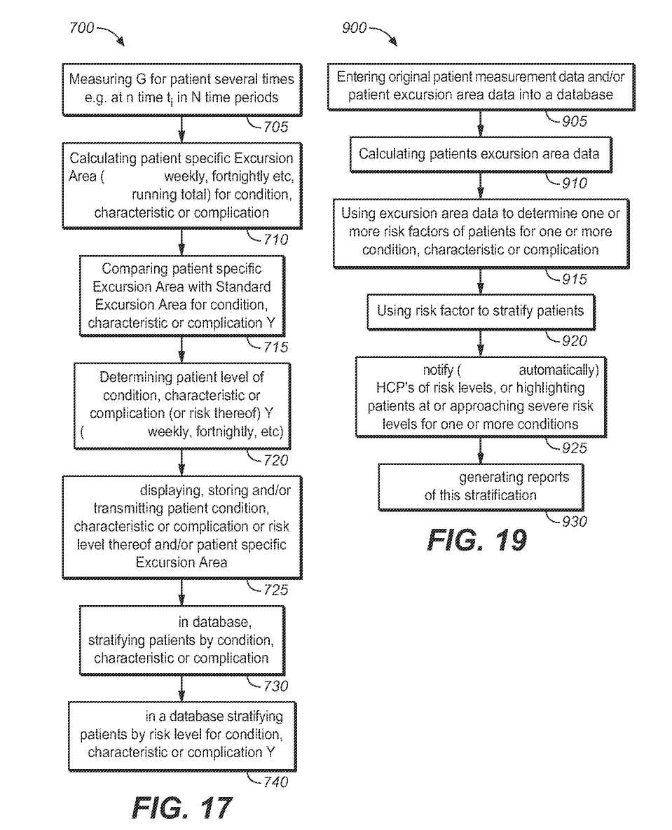

FIG. 17 shows a method 700 for estimating the risk for a specific patient for a specific condition, characteristic or complication hereinafter referred to as condition Y. At step 705 several measurements of analyte are measured for a patient X. At step 710, patient X's specific excursion area is calculated for condition Y (e.g.; above a limit or within certain limits as determined by clinical trial). At step 715 a patient's specific excursion area is compared with a Standard Excursion Area determined from a clinical trial, for condition Y. Next, at step 720 an estimate of the patient risk level of condition Y is determined from comparison at step 715.

Alternatively, at step 725, a patient risk level of condition Y is displayed and/or transmitted and/or stored. Alternatively, at step 730, patients are stratified in a database by condition or characteristic or complication or by risk level.

Similar to that described in relation to FIGS. 13 to 16, it would be possible to establish a ‘Golden’ Standard Excursion Area through, for example, specific clinical trials. This Golden Standard Excursion Area would, in one example embodiment, represent the minimum amount of high-glucose exposure typically required to bring about the onset of diabetes-related complications. The Golden Standard Excursion Area value (similar to the 7% value for HbA1c) would become the benchmark against which the excursion area for each individual is compared for example, weekly, to give an indication of the level of risk of the patient developing diabetes-associated complications.

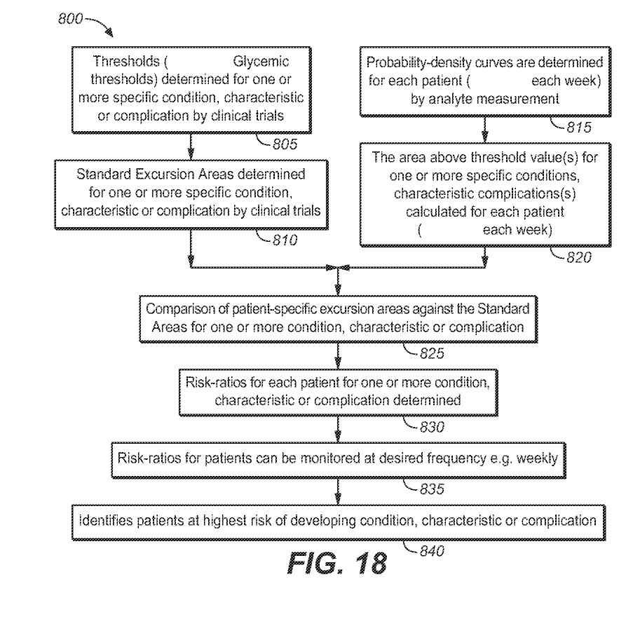

FIG. 18 shows a method of determining risk ratios for several patients in order to identify those at the highest risk of developing complications.

At step 805, one or more threshold limits are determined for one or more specific condition, characteristic or complication by clinical trial. At step 810, Standard Excursion Areas are determined for specific condition, characteristic and/or complication by clinical trial. At step 815 probability density curves are determined for each patient, alternatively, for a certain time period e.g. each week. Alternative time periods are envisaged e.g. fortnight, month, 2 months etc. At step 820, the area above the threshold value for specific complications is calculated for each patient for that time period.

At step 825, comparison of patient-specific excursion areas versus Standard Excursion Areas for each condition, characteristic or complication is carried out to determine a risk ratio, step 830, for each patient for each condition, characteristic or complication. Step 835 indicates monitoring the risk ratios for patients at a desired frequency for example; weekly, fortnightly, monthly, bi-monthly, tri-monthly etc. Step 840 identifies patients at highest risk of developing complications.

FIG. 19 shows a method of stratifying patients 900. At step 905, original patient measurement data and/or excursion area data is entered into a database. If not already calculated, or for completeness sake, a patients excursion area data is calculated at step 910. At step 915 a patient's excursion area data is used to determine a patient's risk factor this is repeated for several patients within the database. Next at step 920 the risk factor is used to stratify patients according to risk.

Alternatively, at step 925 healthcare professionals can be notified of the risk levels of patients e.g. for patients at or approaching severe risk levels. Alternatively, this notification can be automatic. Alternatively, the method can be used to generate a report of the stratification of the patient at step 930.

More specifically in an example embodiment, FIG. 21 shows example probability-density curve depicting a typical probability distribution of blood glucose measurements taken over the period of, for example, one week. Once standard glycemic threshold values pertaining to each type of diabetes-related complication are determined through clinical trials, these may be applied to the probability-density curves obtained for each patient each week. In FIG. 21, 140 mg/dL represents a glycemic threshold value for cardiac complications 84 for example, and 200 mg/dL may represent a threshold value for renal and/or eye complications 86 for example. Other threshold limits may be selected for these or other complications

By way of example only, excursion area 80 shown in FIG. 21 corresponds to a glucose excursion above 140 mg/dL, potentially predicting a high risk of cardiac complications, and excursion area 82 corresponds to the same patient's glucose excursion above 200 mg/dL, potentially predicting a high risk of renal/eye complications. Excursion areas calculated for each patient for each complication would be compared to the known standard excursion areas for each complication, and risk-ratios for each complication can be calculated for each patient each week (as described previously). Using these example values, the excursion area corresponding to cardiac complications could be calculated from the following:

Excursion area (cardiac)=∫₁₄₀⁶⁰⁰f(x)dx

and the excursion area corresponding to renal and/or eye complications could be calculated from the following:

Excursion area (renal/eye)=∫₂₀₀⁶⁰⁰f(x)dx

FIGS. 17 to 19 show flow diagrams of possible steps involved in management of data generated from embodiments of the analytical tool of the present invention, including providing a means of stratifying patients. The present invention provides solutions to the problem of analyzing different forms of real physical data collected from patients and transforming this real physical data into meaningful estimates of the real physical condition of the patients.

It would be useful for healthcare practitioners to have access to a database containing information of how well their diabetic patients are managing to control their disease by means of self-monitoring blood glucose measurements alone. Tight control of their blood glucose concentrations reduces their risk of developing associated complications, can reduce instances of hypoglycemia during either the day or night, and can re-instate warning signs that may have disappeared. Such a database and associated software of the embodiments described herein will enable physicians to work closely with diabetic patients, determining the best method of treatment in each specific case, limit the occurrences of hypoglycemia experienced and allow the diabetic to live as full and normal a lifestyle as possible.

The excursion areas calculated for all diabetic patients belonging to a particular medical practice, hospital, clinic or other specialist establishment, may be entered into and maintained within a central database. The present invention would then allow manipulation and analysis of the information e.g. steps 720, 725, 730, 740, 825, 840 and 905 to 930, 104, enabling the data to be viewed in a number of different ways. An HCP may like to view all data gathered for a particular patient to determine a treatment regime, or they may want to generate an up-to-date list of all patients stratified by risk level for a specific disease or complication. These are only examples of different ways the data could be stratified and viewed; it would be apparent to a person skilled in the art from the information herein that many other different ways of manipulating data would be possible and is not restricted to only those described herein.

The analytical tool of the embodiments described herein provides the ability to generate reports for HCPs, step 930, arming them quickly with an indication of the risk estimates for each patient and/or for each type of complication. The analytical tool may further enable physicians to determine how well individual patients are responding to drugs administered to treat post-prandial blood glucose spikes.

Risk stratification, step 920, provides HCPs with a predictive index of the likelihood of patients developing certain diabetes-related complications. The flexibility of the analytical tools of the invention will allow HCPs to interrogate the data either by disease type or level of risk. They may want to stratify all entries to identify those patients at highest risk, allowing them then to take the necessary precautions such as providing advice, monitoring, further diagnose or treatment of these patients, with highest priority. Patients providing relatively low risk-ratios will then be dealt with afterwards.

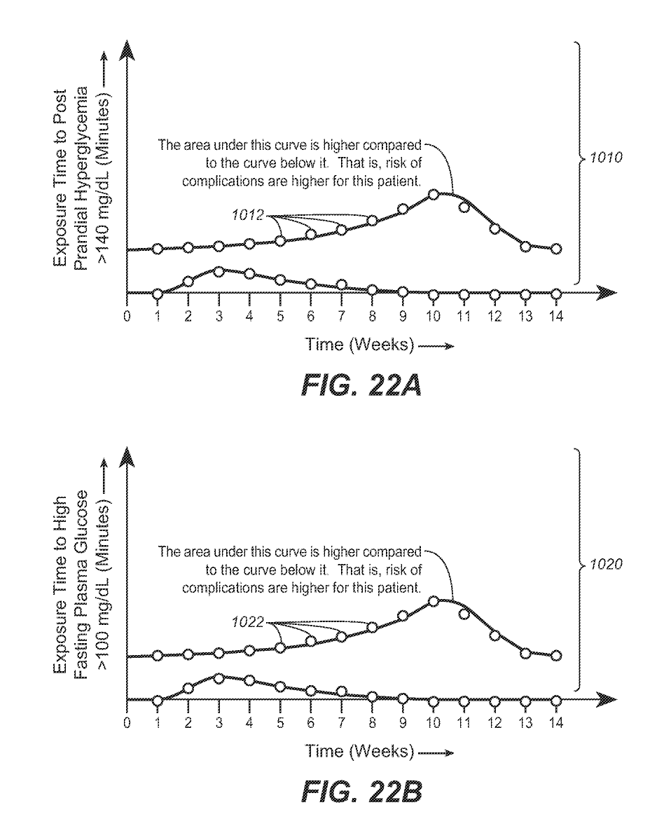

In FIG. 22, a further embodiment is shown. Here, excursion areas (y-axis) for associated glucose concentration measurements for postprandial hypoglycemia over several weeks (x-axis) are shown in graph 1010 having data points 1012. Graph 1020 shows data points 1022 for exposure time to high fasting plasma glucose (of greater than 100 mg/dL) over several weeks. Two patients are shown in each of graphs 1010 and 1020.

Exposure time to high post-prandial glucose is approximated to the area under the curve y=f(G) above threshold limit of 140 mg/dL for selected blood glucose measurements taken post-prandially. This is plotted against time in weeks. Exposure time to high fasting plasma glucose is approximated to the excursion area under the curve y=f(G) above 100 mg/dL for selected blood glucose measurements taken when fasting e.g. pre-meal. This exposure time is plotted against time in weeks.

These exposure times are calculated from probability density curves at the end of each week.

These graphs provide an indication of the frequency and duration of high-glucose excursions previously known to cause complications.

Thus, FIGS. 22A and 22B show example plots of probability-density curves, comparing patients with higher and lower levels of risk of developing complications.

Whether plotting the exposure time of tissue cells to post-prandial hyperglycemia (measurement results>140 mg/dL for example), or the exposure time of tissue cells to high fasting plasma glucose (fasting measurements>100 mg/dL for example), the probability-density curves obtained may appear similar in shape to those shown in FIGS. 22, or these may take a different form. It will be apparent to those skilled in the art that different curve shapes will be expected, and is not restricted to these examples provided herein. These curves may also be used to assist in assessing and stratifying patients according to risk.

Plotting excursion area curves obtained for different patients simultaneously, results in some being positioned higher on the respective y-axis than others. The area under the higher curve will therefore be greater than the area depicted by the lower curve, thereby representing a greater level of risk of developing complications. Such a visual means of displaying numerous results obtained for numerous patients will provide healthcare practitioners with a quick method of identifying those patients most at risk of developing disease-related complications.

It will be apparent to those skilled in the art that analytical tools of the type described herein are intended to provide an aid, particularly to HCPs, as an indicator for use in the identification of patients potentially experiencing high risks associated with the development of known disease-related complications. Such analytical tools are aids to management of a disease in patients.

The analytical tools may also provide pop-up alerts to notify the HCP and/or patient of any data points above or approaching a critical threshold.

Disease management by the method disclosed herein may be a potentially important resource for Health Plans and other means of health insurance. Ways of reducing costs may be possible, by physicians and hospitals focusing on those patients predicted to have the highest risks of developing the most severe disease-related complications. It would be apparent to someone skilled in the art, that decisions regarding management of a disease may require further confirmation tests to be carried out.

One or more of the various analytical tools described herein can be used to compliment the HbA1c test to identify patients that may seem to be in good control, but are actually at risk of complications. Furthermore, one or more of the analytical tools of the invention can be used to assist in stratification of patients by risk level having the same Hb1Ac level. Problems may be identified much quicker using the proposed methods compared to patients having to wait for the results of an HbA1c or 1,5AG test.

The analytical tools of the embodiments described herein may also present an attractive alternative to the HbA1c test and/or the 1,5AG test, both of which require venipuncture.

The analytical tools of the embodiments described herein provide a quick way of determining the extent of fasting and post-prandial excursions for a particular patient. Furthermore, the analytical tools of the embodiments described herein can be used to predict an estimate of the level of risk of a patient developing diabetes-related complications.

One or more embodiments described herein may also be used to determine if a patient is responding properly to drugs particularly useful for evaluating treatment with drugs that target post-meal spikes (or fasting glucose).

One or more of the tools of the embodiments described herein can also be used to reflect acute and transient episodes of hyperglycemia, by depicting a more recent glycemic status in comparison to the HbA1c test. This tool provides an intermediate indicator of glycemic control, and will present patients and their Healthcare Practitioner (HCP) with a more detailed knowledge of glucose excursions, perhaps post meal or when fasting, enabling more timely modifications to their diabetes control regime.

One or more of the analytical tools of the embodiments described herein can also be used to predict whether a patient is more prone to develop one or more complications than another patient with the same A1c value, from self-monitored or continuous blood glucose data alone. Computers adapted to use the tools of the invention may provide HCPs with a prediction of the level of risk of each patient developing specific disease-related complications, and may furthermore provide stratification of multiple patients depending on their risk ratio, allowing efforts to be focused on those with highest risk. This analytical tool may provide an alternative to the HbA1c test.

As noted earlier, the microprocessor can be programmed to generally carry out the steps of various processes described herein. The microprocessor can be part of a particular device, such as, for example, a glucose meter, an insulin pen, an insulin pump, a server, a mobile phone, personal computer, or mobile hand held device. Furthermore, the various methods described herein can be used to generate software codes using off-the-shelf software development tools such as, for example, C, C+, C++, C-Sharp, Visual Studio 6.0, Windows 2000 Server, and SQL Server 2000. The methods, however, may be transformed into other software languages depending on the requirements and the availability of new software languages for coding the methods. Additionally, the various methods described, once transformed into suitable software codes, may be embodied in any computer-readable storage medium that, when executed by a suitable microprocessor or computer, are operable to carry out the steps described in these methods along with any other necessary steps.

Once a patient characteristic has been estimated using any one of the tools of the present invention, an HCP may be able to see in subsequent estimated patient characteristics movement of a patient within a condition, characteristic, complication or risk thereof such as diabetes. For patients grouped into categories such as risk categories this may include movement within or across categories from one group to another. While preferred embodiments of the present invention have been shown and described herein, it will be apparent to those skilled in the art that such embodiments are provided by way of example only. Numerous variations, changes, and substitutions will now occur to those skilled in the art without departing from the invention.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1) A method of analyzing an analyte distribution from discrete, quasi-continuous or continuous measurements comprising:
defining a predetermined time period T;
performing n analyte measurements Gᵢ each associated with a time tᵢ within predetermined time period T with each analyte measurement by a transformation of analyte disposed in body fluid into an enzymatic by-product disposed in body fluid into an enzymatic by-product;
repeating step (b) for N predetermined time periods T;
aggregating the analyte measurements Gᵢ to determine the number of occurrences of each value of Gᵢ across N predetermined time periods T; and
fitting a curve y=f(Gᵢ) to the number of occurrences versus the value of G to determine an estimated probability y=f(G) of the value G occurring within any given predetermined time period Tᵢ.

2) A method of analyzing an analyte distribution from discrete, quasi-continuous or continuous measurements comprising:
defining a predetermined time period T;
providing a measuring device to perform n analyte measurements Gᵢ in a body fluid each associated with a time tᵢ within predetermined time period T with each analyte measurement by a transformation of analyte disposed in body fluid into an enzymatic by-product disposed in body fluid into an enzymatic by-product;
repeating the step of collecting at step (b) for N predetermined time periods T;
providing a microprocessor adapted to aggregate the analyte measurements Gᵢ to determine the number of occurrences of each value of Gᵢ across N predetermined time periods T;
operating the microprocessor to fit a curve y=f(Gᵢ) to the number of occurrences versus the value of G to determine an estimated probability y=f(G) of the value G occurring within any given predetermined time period Tᵢ; and
determining for a user an estimated probability of occurrence of at least one value of G from the fitted curve.

3) The method according to one of claim 1 or 2 in which the step of collecting comprises measuring n analyte measurements Gᵢ at times tᵢ within predetermined time period T.

4) The method according to one of claim 1 or 2 in which the steps of collecting and repeating comprise receiving n analyte measurements Gᵢ taken at times tᵢ within predetermined time period T for N predetermined time periods.

5) The method according to any one of the preceding claims further comprising displaying, storing and/or transmitting the estimated probability of occurrence y=f(G) of at least one value of G.

6) The method according to claim 5 further comprising displaying a numeric value of the estimated probability of occurrence y=f(G) at of least one value of G.

7) The method according to one of the preceding claims further comprising displaying the estimated probability of occurrence of a range of values of G.

8) The method according to one of the preceding claims comprising displaying at least a portion of the fitted curve y=f(G).

9) The method according to any one of claims 1 to 8, further comprising determining a lower limit L₁ and calculating a first excursion area A₁ under a first fitted probability curve above limit L₁ representing the probability of measurement occurring above limit L₁.

10) The method according to claim 9, further comprising determining a Figure of Merit for a patient by:
measuring a corresponding patient characteristic; and
forming a mathematical relationship between the first excursion area A₁ and the measured characteristic.

11) The method according to claim 10 in which the mathematical relationship comprises a product of the first excursion area and the measured characteristic.

12) The method according to any one of the preceding claims further comprising selecting N prior to commencing collecting data.

13) The method according to any one of the preceding claims further comprising displaying, storing and/or transmitting N and/or T.

14) The method according to any one of the preceding claims in which N is increased by 1 after each completed predetermined time period T.

15) The method according to any one of the preceding claims in which N is selected from a group comprising 2, 5, 7, 14, 28, 30, 56, 60, 84, 90, 112, 120 or 240.

16) The method according to any one of the preceding claims in which the predetermined time period T is selected from the group of 1, 2, 3, 4, 6, 12, 24, 48, 72, 96, 168 hours.

17) The method according to any one of the preceding claims in which the step of aggregating to determine the number of occurrences of G is carried out for each range of G including G±ΔG, G+ΔG, G−ΔG.

18) The method according to any one of the preceding claims in which the analyte measurement G comprises a concentration of the analyte.

19) The method according to claim 18 in which the range ΔG is selected from the group of +10, −10, +15, −15, +20, −20, +25, −25, ±10, ±15, ±20, ±25 when G is measured in mg/dl or from the group of +0.1, −0.1, +0.15, −0.15, +0.2, −0.2, +0.25, −0.25, +0.5, −0.5, +0.75, −0.75, +1, −1, ±0.1, ±0.15, ±0.2, ±0.25, ±0.5, ±0.75, ±1 when G is measured in mmol/L.

20) The method according to any one of the preceding claims in which the analyte comprises glucose.

21) The method to claim 20 in which the probability of occurrence of analyte measurement is determined over a range of values of G and the range(s) is/are selected from the group of glucose concentration of less than 100 mg/dL, less than 126 mg/dL, less than 140 mg/dL, less than 200 mg/dL, greater than or equal to 100 mg/dL, greater than or equal to 126 mg/dL, greater than or equal to 140 mg/dL, greater than or equal to 200 mg/dL, greater than or equal to 100 mg/dL and less than 126 mg/dL, greater than or equal to 140 mg/dL and less than 200 mg/dL.

22) A device for analyzing an analyte distribution from discrete, quasi-continuous or continuous measurements comprising:
a collector that obtains analyte measurements Gᵢ;
a microprocessor that receives the analyte measurements, the microprocessor programmed to:
define a predetermined time period T;
collect n analyte measurements Gᵢ each associated with a time tᵢ within predetermined time period T;
repeat step (b) for N predetermined time periods T;
aggregate the analyte measurements Gᵢ to determine the number of occurrences of each value of Gᵢ across N predetermined time periods T;
fit a curve y=f(G) to the number of occurrences versus the value of G to determine an estimated probability y=f(G) of the value G occurring within any given predetermined time period Tᵢ; and
determine for a user an estimated probability or occurrence of at least one value of G from the fitted curve.

23) The device according to claim 22 in which the collector comprises a measuring circuit to measure analyte measurements Gᵢ.

24) The device according to claim 23 in which the collector comprises a receiver to receive analyte measurements Gᵢ from a separate measuring device.

25) The device according to any one of claim 22, 23 or 24 comprising one or more of a display transmitter or memory to display, transmit, or store the estimated probability of occurrence of at least one value of analyte measurement G.

26) The device according to any one of claims 22 to 25 comprising a user interface to receive at least one piece of information and forward the same to the microprocessor.

27) The device according to claim 26 wherein the at least one piece of information is/are selected from the group of i) setting predetermined time period T, ii) updating predetermined time period T, iii) setting N, iv) updating N, v) selecting a portion of the curve y=f(G), vi) selecting a numeric value of at least one probability of a value of G, vii) selecting an excursion area under a probability density curve of G viii) selecting a characteristic estimated from an excursion area ix) selecting one of “IN RANGE”, “OUT OF RANGE” message x) selecting one of “HIGH RISK”, “LOW RISK”, “ACCEPTABLE RISK” messages, xi) selecting display and/or transmission and/or storage of any of the above.

28) The device according to any one of claims 22 to 27, further comprising:
a first component comprising a measurement circuit to measure analyte measurements Gᵢ; and
a second component separate from said first device comprising a microprocessor to receive the analyte measurements and programmed to:
aggregate the analyte measurements Gᵢ to determine the number of occurrences of each value of Gᵢ across N predetermined time periods T;
fit a curve y=f(G) to the number of occurrences versus the value of G to determine an estimated probability y=f(G) of the value G occurring within any given predetermined time period Tᵢ; and
in which said first and second components each comprising a communication circuit for communication therebetween.

29) The device according to claim 28 further comprising one or more of a display, transmitter and/or memory to display, transmit, or store the probability of occurrence of at least one value of analyte measurement G.
